(19) United States
(12) Patent Application Publication
(10) Pub. No.: US 2015/0299806 A1
(43) Pub. Date: Oct. 22, 2015

(54) GENE EXPRESSION PROFILE IN DIAGNOSTICS

(71) Applicant: UNIVERSITY OF TROMSOE, Tromsø (NO)

(72) Inventors: Vanessa Dumeaux, St-Lambert (CA); Eiliv Lund, Tromso (NO)

(73) Assignee: UNIVERSITY OF TROMSOE, Tromsø (NO)

(21) Appl. No.: 14/646,010

(22) PCT Filed: Nov. 20, 2013
(86) PCT No.: PCT/NO2013/050203

§ 371 Date: May 19, 2015

(30) Foreign Application Priority Data
Nov. 20, 2012 (NO) 20121378

(51) Int. Cl. C12Q 1/68

(52) U.S. Cl. CPC C12Q 1/6886; C12Q 2600/158

(57) ABSTRACT

The present invention provides a method for diagnosing, identifying or monitoring proliferative disorders due to e.g cancer preferably breast cancer in a subject by measuring the change of gene expression in a sample e.g. a blood sample. The present invention also encompasses oligonucleotide probes and primers corresponding to genes differentially expressed compared to the expression pattern in a normal cell. The use of such oligonucleotides is also an aspect of the invention together with a kit comprising said oligonucleotides.

### FIELD OF THE INVENTION

The present invention provides a method for diagnosing, identifying or monitoring proliferative disorders due to e.g cancer preferably breast cancer in a subject by measuring the change of gene expression in a sample e.g. a blood sample.

The present invention also encompasses oligonucleotide probes and primers corresponding to genes differentially expressed compared to the expression pattern in a normal cell. The use of the method and the oligonucleotides is also an aspect of the invention together with a kit comprising said oligonucleotides.

### BACKGROUND OF THE INVENTION

Carcinomas or solid tumors are composed of neoplastic epithelial cells, which form the heart of the tumor, as well as a variety of mesenchymal cell types and extracellular matrix components that comprise the tumor stroma, often termed the tumor microenvironment (TME)¹. Historically, it was believed that leukocytic infiltrates, in and around developing neoplasms, were representative of the host's attempt to eradicate neoplastic cells. However, it is now clear that infiltrating immune cells regulate and exert complex, paradoxical roles that are both pro- and anti-tumor during each stage of cancer development².

Recent advances have revealed that tumor-host interactions extend well beyond the local TME (i.e. interactions between the neoplastic cells and the nearby stroma) and that cancer development largely depends on the ability of malignant cells to hijack and exploit the normal physiological processes of the host^{2,3}. Although there is evidence that pronounced abnormalities in a host's systemic immune system caused by aging, stress, immuno-suppressive medications, autoimmune disease or chronic inflammation are associated with increased cancer risk⁴, we lack a precise understanding of how a tumor influences circulating immune cells and vice versa.

Blood pervades the entire body and is in a constant state of renewal⁵. It is the vehicle by which immune cells circulate between central and peripheral lymphoid organs, and migrate to and from tumor sites. Such a crucial role for blood cells as enhancers of tumor physiology raises questions about how they convey their tumor-promoting effects and whether, if understood, it is reflected in the gene expression of circulating cells. Advances in blood RNA processing and transcriptomics allow whole genome gene expression profiling of circulating blood cells⁶. The inventors work within the unique Norwegian Women and Cancer study (NOWAC)⁷ proposed to use transcriptional profiling of cells preferably blood cells to investigate the genesis of cancer preferably breast cancer (BC)⁷ when compared to population-based controls.

The successful treatment of cancer depends in part on early detection and diagnosis. It is however, well established knowledge that a diagnostic method will be dependent on the prevalence of the disease in the population from which the cases (persons diagnosed with the disease) originate.

Unlike gene expression profiles from breast cancer that has been reported previously^{49,50} which were determined using cases and control group based on what is called a “workout” which means that the tests are based on control groups with positive findings (abnormal mammograms) and the prevalence of breast cancer in the populations involved will be about 50%, while the prevalence using samples from the control group of the present invention wherein population-based controls from the NOWAC cohort (healthy controls matched by time of follow-up and birth year within the cohort) were studied will be less than 0.5‰.

It is believed that the method according to the present invention will be a more reliable diagnostic method, as the gene expression profile has been built up as a result of genes differentially expressed in samples from cases compared to samples from healthy people from the population wherein the test is to be used. It is therefore an objection of the present invention to provide an in vitro method for diagnosing, identifying or monitoring proliferative disorder in a subject, preferably a human being.

The differences in the control groups as described above between WO 2011/086174 A2 and Aarøe et al. and the present invention may explain the differences between the identified gene profiles.

It should also be noted that our list is based on a study run as an epidemiological study and not with the potentials for quality controls as in a clinical setting. This is an important aspect for improving the test.

The inventors have disclosed how the presence of a proliferative disorder are reflected in the gene expression of the host's cells, preferably circulating blood cells, and further how to utilize this in diagnosing, identifying or monitoring proliferative disorder e.g. cancer, preferably breast cancer. It has further been shown that specific functional “groups” of genes are specifically expressed. The method of the present invention may be able to detect the expression pattern reflecting a proliferative disorder long before the onset of symptoms appear.

The primary benefit of breast cancer screening is early detection, which typically results in simpler treatment, a lower chance of recurrence and a greater chance of survival. For younger woman, sensitivity of mammography screening can be as low as 58% and false negatives results cause a delay in diagnosis for women who think they are safe of breast cancer. The cumulative risk of false positive reading after 10 mammographic screens ranges 21% to 49% for all women. In population offered mammography screening, the risk of unnecessary surgeries was increased by 31% compared to women not using mammography.

More specific technologies complementing existing screening will clearly be beneficial and we believe that the present invention providing a robust new diagnostic method may be such a complement. The method may however, further be used separately for diagnosing, identifying or monitoring proliferative disorder without being part of a screening program.

### SUMMARY OF THE INVENTION

The present invention encompasses in first aspect, an in vitro method for diagnosing, identifying or monitoring proliferative disorder in a subject, which method comprises the following step:

a) measuring the level of gene expression in a subset of genes set forth in Table 1 or 2 in a sample from said subject;

b) comparing the level of gene expression of the subset of genes in the sample from said subject with the level of gene expression of the subset of genes in a standard gene expression pattern extracted from healthy subjects;

c) wherein change of gene expression of each and one gene of said subset in said sample as compared to a standard gene expression pattern being indicative for a proliferative disorder.

A second aspect of the present invention comprises a set of oligonucleotide probes, wherein said set is selected from the oligonucleotides of Table 1 or 2 or oligonucleotides derived from a sequence set forth in Table 1 or 2 or any combination thereof, or a oligonucleotide with a complementary sequence, or a functional equivalent thereof.

In a further aspect the invention comprises a kit for in vitro diagnosing, identifying or monitoring proliferative disorder comprising:

a collection of oligonucleotide probes and primers capable of detecting the level of expression of at least about 50, preferably at least about 30, more preferably at least about 20, most preferably at least 10 and even more preferably at least 2 forth in Table 1 or 2 or any combination thereof.

The present invention also provides, in one aspect use of the method as described, or the set of oligonucleotide probes, or the kit for diagnosing, identifying or monitoring proliferative disorder in a subject.

Preferred embodiments are set forth in the dependent claims and in the detailed description of the invention.

### DESCRIPTION OF THE FIGURES

FIG. 1. Gene expression changes in circulating blood cells of breast cancer patients compared to controls.

A. Venn diagram depicting the overlap between genes differentially expressed in circulating blood cells of breast cancer patients compared to controls in the primary (CC1) and the secondary (CC2) dataset. Differential expression was assessed at an FDR<0.005 in the paired linear analyses. B. Expression fold changes for the 345 overlapping genes differentially expressed in CC1 and CC2. Log fold-changes (log FC) in CC1 are plotted on the x-axis against the log FCs for the same genes in CC2 on the y-axis. Light grey, genes downregulated in the breast cancer group in both data sets; dark grey genes upregulated in the breast cancer group in both data sets. C. Ordering of blood samples according to the sum of expression over those 345 overlapping genes. Heat map colors represent mean-centered fold change expression in log-space D. Accuracy significances of predictors in the validation dataset (CC3). Vertical line represents the accuracy significance of a predictor based on the expression of the 345 overlapping genes. The stippled line represents the distribution of accuracy significances that can be obtained from 100,000 predictors built using 345 random genes present in CC3 (N=8529). The dotted line represents the distribution of accuracy significances that can be obtained from 100,000 predictors built using 50 genes among the 341 expressed in CC3 of the 345 overlapping genes. Plane line represents the distribution of accuracy significances that can be obtained from 100,000 predictors built using 50 random genes present in the dataset (N=8529).

FIG. 2. Ordering of blood samples based on the enrichment scores of the 38 significant gene sets differentially expressed between breast cancer cases (in dark grey) and controls (in light grey) in the primary (CC1), secondary (CC2) and validation (CC3) case-control series. Heat map colors represent mean-centered fold change enrichment score in log-space.

FIG. 3. Gene set variation analysis of the antigen processing and presentation pathway and the natural killer cell gene set.

A. The antigen processing and presentation pathway from KEGG. Overlapping core genes driving the observed association of the Ag processing and presentation pathway with the presence of breast cancer within CC1 and CC2 are colored according to their up- (dark grey) or down- (light grey) regulation in breast cancer patients.

B. Boxplot indicating the enrichment scores from gene set variation analysis for the natural killer (NK) cell gene set associated with the gene expression profiles from breast patients (dark grey) and controls (light grey) included in CC1, CC2 and CC3 (left). List of genes included in the NK gene set significantly associated to disease status in paired linear analysis with FDR<0.10 in at least two of the three datasets and their corresponding median fold-changes over all datasets (right).

FIG. 4. Accuracy significances of predictors in the validation dataset (CC3) that can be obtained from 100,000 predictors built using 10, 25 or 50 genes among the 341 expressed in CC3 of the 345 overlapping genes.

FIG. 5. Ordering of blood samples (dark grey: cases, light grey: controls) based on the top quintile of most variable genes in the primary case-control series (CC1). Heat map colors represent mean-centered fold-change expression in log-space.

FIG. 6. Accuracy significances obtained to predict breast cancer diagnosis in the publicly available dataset including gene expression profiles of peripheral blood mononuclear cells (PBMC) from BC patients, pre- and post-surgery samples, patients from benign breast diseases, controls, gastrointestinal and brain cancer patients (GSE27562). Vertical lines represent the accuracy significances of a predictor based on the expression of the 49 genes present in the dataset among our 50-gene best predictor list to predict breast cancer case from controls (plain line) and to predict breast cancer or benign breast abnormalities cases from controls (dotted line). The distribution curves represent the respective distribution of accuracy significances that can be obtained from 100,000 predictors built using 49 random genes present in CC3 (N=8242).

FIG. 7. Accuracy significances obtained to predict breast cancer diagnosis in the publicly available dataset including gene expression profiles of whole blood cells from BC patients and controls with suspect mammograms. Vertical lines represent the accuracy significances of a predictor based on the expression of the 32 genes present in the dataset among our 50-gene best predictor list (dotted line) and of the 63 genes present plain line). The dotted distribution curve represents the distribution of accuracy significances that can be obtained from 100,000 predictors built using 32 random genes present in CC3 (N=8242).

FIG. 8. Covariate plot displaying the individual contributions of each gene in the antigen processing and presentation pathway to the overall test statistics for differential expression in the primary (CC1) and secondary (CC2) case-control series. Significantly contributing genes (p<0.1) are indicated by the dark line in the hierarchical tree. The grey and the black colors represent association with the control and the breast cancer cases groups, respectively.

### DETAILED DESCRIPTION OF THE INVENTION

It is an object of the present invention to provide a method for diagnosing, identifying or monitoring proliferative disorders due to e.g cancer preferably breast cancer in a subject by measuring the change of gene expression in a sample e.g. cells from a blood sample. The present invention also encompasses oligonucleotide probes and primers corresponding to genes differentially expressed compared to the expression pattern in a normal cell. The use of the method and oligonucleotides is also an aspect of the invention together with a kit comprising said oligonucleotides.

Norway has around 436,338 women born between 1943 and 1957, of whom 148,536 (34.4%) participate in the Norwegian Women and Cancer study (NOWAC)^{6,8,40}. This unique material formed the basis of the study resulting in the present invention.

Blood samples were collected at time of diagnosis. The inventors first selected 96 blood samples from breast cancer patients and 116 blood samples from healthy controls matched by time of follow-up and birth year within the NOWAC cohort. From each sample, RNA was extracted from whole blood, and one control sample of two with the highest RNA quality and quantity was then amplified and hybridised along with its corresponding case sample. Using Illumina microarrays, whole blood gene-expression profiles for 96 breast cancer cases and 96 matched controls were generated. Probes fluorescence intensities of scanned images were quantified, trimmed, averaged and normalized to yield the transcript abundance of a gene as an intensity value.

In this primary dataset (CC1), some 9,338 unique genes were regulated across the group of 55 pairs of case/control. The inventors investigated blood gene expression profiles from an additional 63 pairs of BC cases and controls processed in the same way than CC1. To test whether these data findings where replicable in an independent data set a secondary dataset (CC2) was tested. After preprocessing of this secondary dataset (CC2), some 7898 unique genes and 49 pairs of case-control were included in the analyses. 818 of the 7898 genes passing the quality controls were differentially expressed in CC2, of which 345 were also differentially expressed in CC1. Remarkably, the directionality of differential expression between breast cancer cases and control of all 345 overlapping genes was conserved between the datasets. When patients were ranked according to the sum of expression over the 345 overlapping genes, the majority of blood samples from breast cancer cases were segregated from controls in both dataset.

These findings indicate that a basis of a reliable method for diagnosing, identifying and monitoring a proliferative disorder e.g cancer, preferably breast cancer, has been established. The overlapping 345 genes and alternative splice variants of some of said genes presented in Table 1 may serve as a source from where a subset of genes is chosen and the level of expression is measured and compared to a standard expression pattern. Also oligonucleotide probes and primers may be generated. A selection of preferred 50 genes is presented in Table 2. Each gene in Table 1 and 2 are denoted by international recognized gene symbols.

In order to validate the findings in CC1 and CC2 the validation dataset (CC3) consisted in 8529 unique genes expressed across 59 pairs of breast cancer cases and controls was investigated. Microarray data have been deposited at European Genome-phenome Archive (EGA; https://www.ebi.ac.uk/ega/).

In the 345-gene list, most differentially expressed transcription factors and genes that are part of the general transcription machinery (Table 4) are in blood cells from breast cancer patients compared to controls suggesting that down regulated steady state transcription rates are reduced.

Coordinate and sharp waves of expression of large sets of genes in response to a common stimulus imply that a broad regulatory mechanism is in place, possibly in response to the presence of breast cancer. Although transcription is an essential first step in the regulation of gene expression, the expression of key factors controlling immune cell differentiation and/or function are regulated at the translational level¹⁵. In our study, post-transcriptional regulons based on RNA binding proteins (RBPs) were identified as having essential roles in the control of blood cell gene expression changes associated with breast cancer (Table 4, Table 5). RBPs organize nascent RNA transcripts into groups in order to percolate them together down the chain of splicing, nuclear export, stability and translation so that proteins are efficiently produced to meet the needs of the cell¹⁶. The abundance of genes in the immune system that are alternatively spliced¹⁷, and the connections between splicing and disease in our study, imply that alternative splicing may be a crucial mechanism for regulating and fine-tuning the function of the circulating blood cells associated with the presence of breast cancer.

The inventors surprisingly detected that circulating blood cells in breast cancer patients are enriched in genes which are associated with, and may depend on, systemic immunosuppression, cell motility, metabolism and proliferation. These findings may further be determining when selecting the subset of genes and/or oligonucleotides or primers of the present invention.

In conclusion, the gene expression of circulating blood cells is markedly perturbed in a specific pattern by irregularities other places in the body as e.g the presence of cancer, preferably breast cancer. These detectable changes form a reliable specific pattern not seen in blood cells form healthy persons and will serve as an excellent platform for diagnostic, identification or monitoring purposes. As a consequence the proliferative disorder may be detected long before the onset of other symptoms appears.

The blood-based gene expression method according to the present invention produced surprisingly uniquely robust and reproducible results across microarray platforms and external datasets to detect proliferative disorder, e.g. cancer, preferably breast cancer from population-based controls.

Thus the invention comprises a method for diagnosing, identifying or monitoring proliferative disorders due to e.g. cancer, preferably breast cancer in a subject by measuring a change in gene expression in cells from a sample e.g. a blood sample.

Accordingly a first aspect of the present invention relates to an in vitro method for diagnosing, identifying or monitoring proliferative disorder in a subject, which method comprises the following step:

a) measuring the level of gene expression in a subset of genes set forth in Table 1 or 2 in a sample from said subject;

b) comparing the level of gene expression of the subset of genes in the sample from said subject with the level of gene expression of the subset of genes in a standard gene expression pattern extracted from healthy subjects; and

c) change of gene expression of each and one gene of said subset in said sample as compared to a standard gene expression pattern being indicative for a proliferative disorder.

As used herein, “proliferative disorder”, refers to a condition where cells are produced in excessive quantities as e.g. in cancer.

As used here in “subject” refers to a mammal. The term “subject” therefore includes for example primates (e.g. humans) and e.g. animals as cows, sheep goats, horses, dogs, cats.

As used here in “sample” refers to a biological sample (e.g. blood sample or fluid sample)

As used herein, “gene expression” refers to translation of information encoded in a gene into a gene product (e.g., RNA, protein). Expressed genes include genes that are transcribed into RNA (e.g., mRNA) that is subsequently translated into protein as well as genes that are transcribed into non-coding functional RNA that are not translated into protein (e.g., tRNA, rRNA ribozymes etc.)

As used herein “level of gene expression” or “expression level” refers to the level (e.g., amount) of one or more products (e.g. RNA, protein) encoded by a given gene in a sample or reference standard.

As used herein “subset of genes” refers to a combination of two or more genes, each of which display a change in the expression pattern relative to the standard gene expression pattern. The subset of genes may be selected from the list of Table for 2 or any combinations thereof.

In one embodiment the subset of genes does not contain the gene: ABHD10

In one embodiment the subset of genes does not contain the gene: AB13

In one embodiment the subset of genes does not contain the gene: ANXA1

In one embodiment the subset of genes does not contain the gene: AQP9

In one embodiment the subset of genes does not contain the gene: C14orf2

In one embodiment the subset of genes does not contain the gene: CLN5

In one embodiment the subset of genes does not contain the gene: ELA C2

In one embodiment the subset of genes does not contain the gene: EXOC6

In one embodiment the subset of genes does not contain the gene: EXCOSC10

In one embodiment the subset of genes does not contain the gene: GMFG

In one embodiment the subset of genes does not contain the gene: GPBAR1

In one embodiment the subset of genes does not contain the gene: GPR68

In one embodiment the subset of genes does not contain the gene: HIST1H2BK

In one embodiment the subset of genes does not contain the gene: HSPBAP1

In one embodiment the subset of genes does not contain the gene: KIF13B

In one embodiment the subset of genes does not contain the gene: RPL4

In one embodiment the subset of genes does not contain the gene: RPL15

In one embodiment the subset of genes does not contain the gene: RPS29

In one embodiment the subset of genes does not contain the gene: RPL7

In one embodiment the subset of genes does not contain the gene: H2AFX

In one embodiment the subset of genes does not contain the gene: MAPREI

In one embodiment the subset of genes does not contain the gene: NUP62

In one embodiment the subset of genes does not contain the gene: PGAM1

In one embodiment the subset of genes does not contain the gene: PLAGL2

In one embodiment the subset of genes does not contain the gene: RELA

In one embodiment the subset of genes does not contain the gene: RNH1

In one embodiment the subset of genes does not contain the gene: RPL11

In one embodiment the subset of genes does not contain the gene: RPL15

In one embodiment the subset of genes does not contain the gene: RPL21

In one embodiment the subset of genes does not contain the gene: RPS3A

In one embodiment the subset of genes does not contain the gene: S100A8

In one embodiment the subset of genes does not contain the gene: TBC1D15

In one embodiment the subset of genes does not contain the gene: THOC4

In one embodiment the subset of genes does not contain the gene: VPS52

In one embodiment the subset of genes does not contain the gene: YWHAB

In one embodiment the set of oligonucleotide probes does not contain SEQ ID NO:2

In one embodiment the set of oligonucleotide probes does not contain SEQ ID NO:3

In one embodiment the set of oligonucleotide probes does not contain SEQ ID NO:20

In one embodiment the set of oligonucleotide probes does not contain SEQ ID NO:33

In one embodiment the set of oligonucleotide probes does not contain SEQ ID NO:58

In one embodiment the set of oligonucleotide probes does not contain SEQ ID NO:96

In one embodiment the set of oligonucleotide probes does not contain SEQ ID NO:165

In one embodiment the set of oligonucleotide probes does not contain SEQ ID NO:176

In one embodiment the set of oligonucleotide probes does not contain SEQ ID NO:181

In one embodiment the set of oligonucleotide probes does not contain SEQ ID NO:182

In one embodiment the set of oligonucleotide probes does not contain SEQ ID NO:209

In one embodiment the set of oligonucleotide probes does not contain SEQ ID NO:214

In one embodiment the set of oligonucleotide probes does not contain SEQ ID NO:215

In one embodiment the set of oligonucleotide probes does not contain SEQ ID NO:220

In one embodiment the set of oligonucleotide probes does not contain SEQ ID NO:228

In one embodiment the set of oligonucleotide probes does not contain SEQ ID NO:229

In one embodiment the set of oligonucleotide probes does not contain SEQ ID NO:251

In one embodiment the set of oligonucleotide probes does not contain SEQ ID NO:273

In one embodiment the set of oligonucleotide probes does not contain SEQ ID NO:280

In one embodiment the set of oligonucleotide probes does not contain SEQ ID NO:283

In one embodiment the set of oligonucleotide probes does not contain SEQ ID NO:292

In one embodiment the set of oligonucleotide probes does not contain SEQ ID NO:293

In one embodiment the set of oligonucleotide probes does not contain SEQ ID NO:294

In one embodiment the set of oligonucleotide probes does not contain SEQ ID NO:296

In one embodiment the set of oligonucleotide probes does not contain SEQ ID NO:297

In one embodiment the set of oligonucleotide probes does not contain SEQ ID NO:300

In one embodiment the set of oligonucleotide probes does not contain SEQ ID NO:311

In one embodiment the set of oligonucleotide probes does not contain SEQ ID NO:336

In one embodiment the set of oligonucleotide probes does not contain SEQ ID NO:346

In one embodiment the set of oligonucleotide probes does not contain SEQ ID NO:347

In one embodiment the set of oligonucleotide probes does not contain SEQ ID NO:353

In one embodiment the set of oligonucleotide probes does not contain SEQ ID NO:394

In one embodiment the set of oligonucleotide probes does not contain SEQ ID NO:406

In one embodiment the set of oligonucleotide probes does not contain SEQ ID NO:407

In one embodiment the set of oligonucleotide probes does not contain SEQ ID NO:409

In one embodiment the set of oligonucleotide probes does not contain SEQ ID NO:410

In one embodiment the set of oligonucleotide probes does not contain SEQ ID NO:412

In one embodiment the set of oligonucleotide probes does not contain SEQ ID NO:413

In one embodiment the set of oligonucleotide probes does not contain SEQ ID NO:414

In one embodiment the set of oligonucleotide probes does not contain SEQ ID NO:419

In one embodiment the set of oligonucleotide probes does not contain SEQ ID NO:420

In one embodiment the set of oligonucleotide probes does not contain SEQ ID NO:421

In one embodiment the set of oligonucleotide probes does not contain SEQ ID NO:422

In one embodiment the set of oligonucleotide probes does not contain SEQ ID NO:423

In one embodiment the set of oligonucleotide probes does not contain SEQ ID NO:424

In one embodiment the set of oligonucleotide probes does not contain SEQ ID NO:425

In one embodiment the set of oligonucleotide probes does not contain SEQ ID NO:434

In one embodiment the set of oligonucleotide probes does not contain SEQ ID NO:479

In one embodiment the set of oligonucleotide probes does not contain SEQ ID NO:487

In one embodiment the set of oligonucleotide probes does not contain SEQ ID NO:522

In one embodiment the set of oligonucleotide probes does not contain SEQ ID NO:532

In one embodiment the set of oligonucleotide probes does not contain SEQ ID NO:533

As used herein “healthy subject” refers to a subject from the NOWAC cohort representing population-based controls (healthy controls matched by time of follow-up and birth year within the cohort).

In one embodiment of this aspect the subset of genes may at least be 50, preferably at least 30, more preferably at least 20, most preferably at least 10 and even more preferably at least 2 or any combination thereof.

In one embodiment the expression from all 50 genes of Table 2 may be measured.

In a further embodiment the expression from all 345 genes of Table 1 may be measured.

In yet a further embodiment only one gene of Table 1 or 2 may be measured.

In a further embodiment the subset of genes may be associated with systemic immunosuppression, cell motility, metabolism and/or proliferation as set forth in Table 1 or 2 or any combination thereof. Also other functional groups of genes may constitute a subset of genes.

In yet a further embodiment the change of gene expression is measured to be at least 10%, at least 20%, at least 30% when measured as intensity value of scanned images. Other detection methods may be but are not limited to e.g. PCR.

In one or more embodiments of the method of the present invention the difference in gene expression may be measured by using oligonucleotide probes selected from a group set forth in Table 1 or 2, said oligonucleotide probes may be derived from a sequence set forth in Table 1 or 2, or may further be complementary to the sequence of the oligonucleotides set forth in Table 1 or 2, or be functional equivalent to the oligonucleotides of Table 1 or 2. The oligonucleotide may act as a detectable probe targeting the correspondent target sequence or may be used as a primer in the amplification step described in the Example section. The subset of genes, oligonucleotides probes or primers according to the present invention may be selected from Table 1 or 2 or any combination thereof.

In a further embodiment said oligonucleotide probes is a set of probes of at least about 50, preferably at least about 30, more preferably at least about 20, most preferably at least about 10 and most preferably at least about 2 oligonucleotide probes.

As used herein “oligonucleotide” refers to a nucleic acid molecule and may be DNA, RNA or PNA (peptide nucleic acid) or hybrids thereof or modified forms thereof, e.g chemically modified by e.g methylation. An oligonucleotide probe according to the invention have the ability to bind and probe complementary sequences in the target gene or gene product.

As used herein “derived” refers to oligonucleotides derived from the genes corresponding to the sequences set forth in Table 1 or 2. As the Tables provides Illumina identifiers and internationally recognized HUGO gene symbols, the probe or the primer sequences may be derived from anywhere on the gene to allow specific binding to that gene or its transcript.

As used herein “complementary sequences” refers to sequences with consecutive complementary bases, making it possible for the sequences to bind to one another through their complementarity.

As used herein “functional equivalent” refers to a oligonucleotide which is capable of identifying and binding to the transcript (or DNA) of the same gene as the oligonucleotides set forth in Table 1 or 2 or sequences derived from Table 1 or 2.

In a further embodiment the oligonucleotide may be a probe or a primer which hybridizes under stringent conditions, preferably under high stringency conditions. In further embodiments the oligonucleotide probe may be immobilized on one or more solid supports, which may be but are not limited to e.g a membrane, a plate or a biochip.

As used herein “hybridize under high stringency condition” refers to a specific association of two complementary nucleotide sequences (e.g., DNA, RNA or a combination thereof) in a duplex under stringent conditions as a result of hydrogen bonding between complementary base pairs. Under high stringency conditions do not permit hybridization of two nucleic acid molecules that are not complementary (two nucleic acids molecules that have less than 70% sequence complementarities).

In yet another embodiment said oligonucleotides or primers may preferably be at least about 20, 50, 100 or 200 nucleotides in length.

In further embodiments of the present invention the levels of gene expression in said subset of genes may be detected in said sample by determining the levels of RNA molecules encoded by said genes. The levels may but are not restricted to be determined by the use of microarray technique. Also other detection methods as e.g. PCR may be used.

As used herein “RNA” refers to total RNA in a cell.

In another embodiment the sample wherein the gene expression is measured may be circulating blood cells e.g from whole blood, peripheral blood mononuclear cells or other subsets of blood cells. Also cells from body fluids or other tissues may be a sample source as the inventors surprisingly detected that the expression profile of differentially expressed genes found in blood cells also were seen in circulating mononuclear cells including monocytes, T-cells and natural killers cells. In one embodiments these cells may isolated and used as a sample source of the present invention.

In further embodiments the proliferative disorder may be breast cancer and the subject may be but is not restricted to a human being.

In one embodiment the detection of differentially expressed genes may be obtained by translating the subset of genes from Table 1 or 2 to PCR technology and identify the genes differentially expressed compared to control genes.

In another embodiment the detection may be to identify every pair of genes in the subset of genes from Table 1 or 2 and identify the differentially expressed genes.

I yet another embodiment the pair of genes in the subset of genes of Table 1 or 2 may be investigated and rules created as e.g. if gene 1>gene 2 etc. then it's a positive case.

In order to decide if a sample from a subject reflects the specific expression pattern characteristic of a proliferative disorder as e.g. cancer, preferably breast cancer, a standard gene expression pattern may be provided, one embodiment of the present invention comprises an in vitro method for preparing a standard gene expression pattern reflecting proliferative disorder in a subject, which method comprises the following step:

a) measuring the level of gene expression in a sample from said subject,

b) measuring level of gene expression in a control sample from a healthy subject;

c) comparing level of gene expression of the sample from said subject with the level of gene expression in a control sample from the healthy subject to produce a characteristic standard gene expression pattern from genes reflecting proliferative disorder as set forth in Table 1 or 2.

According to a second aspect the present invention relates to a set of oligonucleotide probes, wherein said set may be selected from the oligonucleotides of Table 1 or 2 or oligonucleotides derived from a sequence set forth in Table 1 or 2 or a oligonucleotide with a complementary sequence, or a functional equivalent oligonucleotide.

In a further embodiment the set of oligonucleotide probes may comprise at least about 50, preferably at least about 30, more preferably at least about 20, most preferably at least about 10 and most preferably at least about 2 oligonucleotide probes.

The oligonucleotide may act as a detectable probe targeting the correspondent target sequence or may be used as a primer in the amplification step described in the Example section. The oligonucleotide probes or primers according to the present invention may be selected from a group of Table 1 or 2 or any combination thereof.

In one or more embodiments a set of oligonucleotide probes may hybridize under high or medium stringency conditions with the corresponding subset of genes of Table 1 or 2 Further the oligonucleotide probes may be immobilized on a solid support, which may be but are not limited to e.g a membrane, a plate or a biochip.

The use of the oligonucleotides in a product. e.g. a kit form further aspects of the present invention.

Accordingly a further aspect of the present invention relates to a kit for in vitro diagnosing, identifying or monitoring a proliferative disorder comprising:

a collection of oligonucleotide probes and/or primers capable of detecting the level of expression of at least about 50, preferably at least about 30, more preferably at least about 20, most preferably at least 10 and even more preferably at least 2 genes selected from the group set forth in Table 1 or 2. The collection of oligonucleotides provided in the kit of the present invention may be selected from a group or any combination thereof according to Table 1 or 2.

In another embodiment the said probes may specifically hybridize to RNA transcripts of said genes, further the kit may comprise a set of oligonucleotide probes as defined above.

In further embodiments the oligonucleotide probes are preferably immobilized on one or more a solid supports wherein the solid support may be e.g a membrane, a plate, or a biochip.

The present invention also provides, in one aspect use of the method as described, or the set of oligonucleotide probes, or the kit for diagnosing, identifying or monitoring proliferative disorder in a subject.

Having now fully described the present invention in some detail by way of illustration and example for purpose of clarity of understanding, it will be obvious to one of ordinary skill in the art that same can be performed by modifying or changing the invention by with a wide and equivalent range of conditions and other parameters thereof, and that such modifications or changes are intended to be encompassed within the scope of the appended claims.

### EXAMPLES

### Example 1

### Blood-Wide Transcriptional Signal of Breast Cancer and its Diagnostic Potential

NOWAC is a prospective cohort that follows a large representative sample of the Norwegian female population (˜34% of all women born between 1943-1957) and biobanks blood samples both prior to (n=50,000), and at the time of, breast cancer diagnosis (n=385 cases with age-matched controls)⁸. We selected from this cohort 96 blood samples from breast cancer patients and 116 blood samples from controls matched by time of follow-up and birth year. From each case sample, total RNA was extracted from whole blood, and a matched control sample was selected that exhibited the highest quality and quantity of RNA. Both case and control were amplified and hybridized simultaneously. to ablate technical effects using Illumina microarrays. In total, we generated whole blood gene-expression profiles for 96 breast cancer cases and 96 matched controls. Samples received more than 4 days after collection (N=6), with low RNA quality (RIN<7, N=24), outliers (N=14), misdiagnosis (N=3) and unmatched samples (N=35) were excluded from our analyses. Probes intensities of scanned images were quantified, trimmed, normalized and averaged to yield the transcript abundance of each gene as an intensity value. In this first case-control dataset (CC1), these quality control steps identified 9,338 unique genes of sufficient quality across a group of 55 pairs of case/control (Table 3). Disease status was associated with substantial differences in blood gene expression profiles across the case/control pairs included in CC1 (P=6×10⁻⁸, global test. This blood-wide signal of breast cancer diagnosis is illustrated by the grouping of samples according to disease status in an unsupervised clustering based on the top quintile of most variable genes (FIG. 5). We identified 3479 genes showing significant differences in expression (False Discovery Rate (FDR)<0.005; paired linear analysis) between the breast cancer and controls groups with a relatively low median absolute value of fold-change equal to 1.13 and ranging from 1.03 to 2.03. This indicates that gene expression changes associated with breast cancer are of relatively low amplitude but ubiquitous in circulating blood cells.

### Data Analysis

The data analysis in the examples was performed using R (http://cran.r-project.org), an open-source-interpreted computer language for statistical computation and graphics, and tools from the Bioconductor project (http://www.bioconductor.org), adapted to our needs. To identify single gene differentially expressed between cases and controls, we conducted paired gene-wise linear analysis with application of empirical Bayes methods implemented in the software package Limma for the R computing environment. False discovery rate⁴¹ (FDR) was calculated to adjust for multiple testing. We trained naive Bayes' classifiers to predict outcome using the ranked log-odds that the gene is differentially expressed in the paired linear analysis and used internal leave-one-out cross-validation. We investigated the distribution of accuracy significances that can be obtained from 100,000 predictors built using a defined number of genes among the selected gene list, and compared it with predictors using the same number of genes randomly chosen within the dataset. Functional annotations were curated from the GeneCards encyclopedia⁴² (www.genecards.org). We applied functional clustering via the Database for Annotation, Visualization, and Integrated Discovery (DAVID) (ref) (http://david.abcc.ncifcrf.gov/). Gene set variation analysis package for the R computing environment implements a non-parametric unsupervised method for assessing gene set enrichment in each sample. We used several compendia and ontologies including the Gene Ontology⁴³ (GO), the KEGG⁴⁴ and Biocarta (http://www.biocarta.com/) pathway databases, and curated gene signatures databases obtained from the literature, all available from the Molecular Signature Database, MSigDB⁴⁵. Gene set specific to immune cell subsets were generated using CD markers of no more than 3 immune cell subtypes²¹ and transcripts specifically overexpressed in each differentiated immune cell subtype²². The global test {Goeman, 2004 24/id} was used to test the influence of groups of genes on pairs of breast cancer cases and controls. Genes or cluster of genes driving the observed association of the gene set with the presence of BREAST CANCER were defined as core genes (multiplicity corrected P-value<0.1).

### Example 2

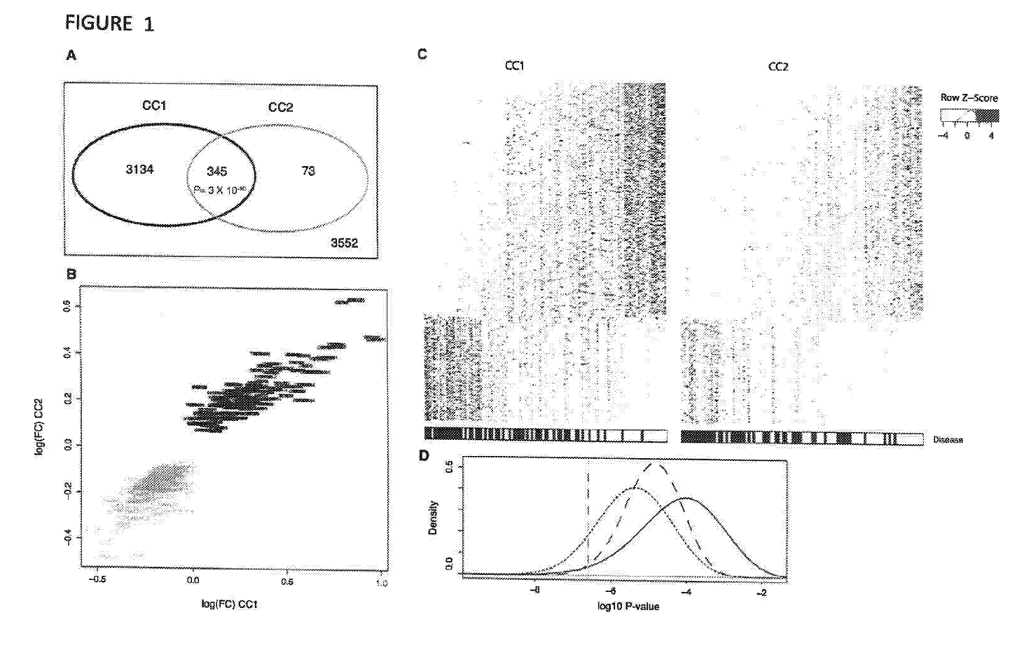

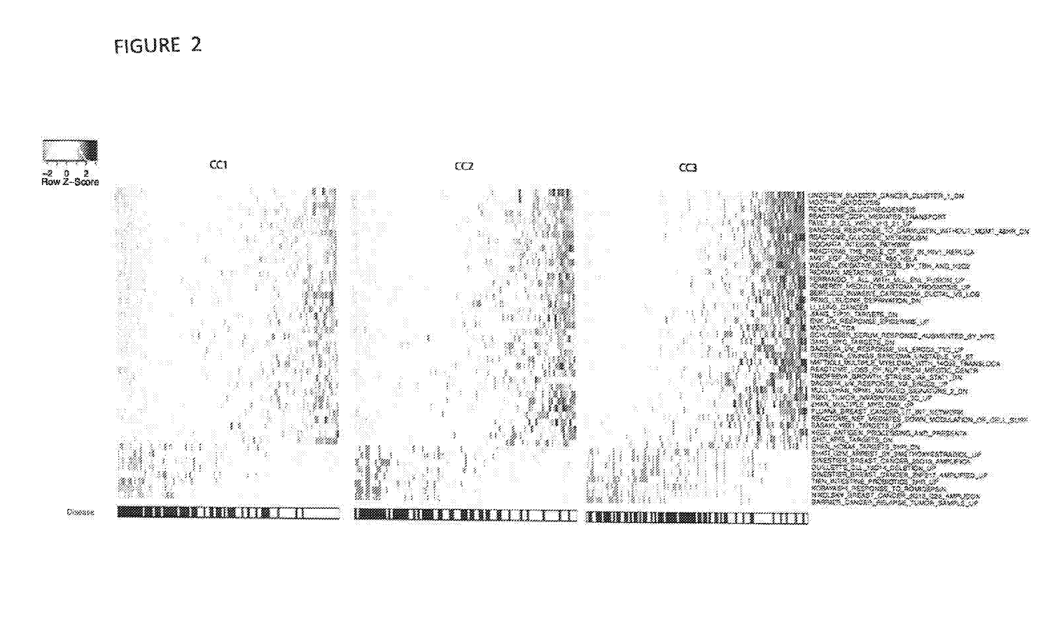

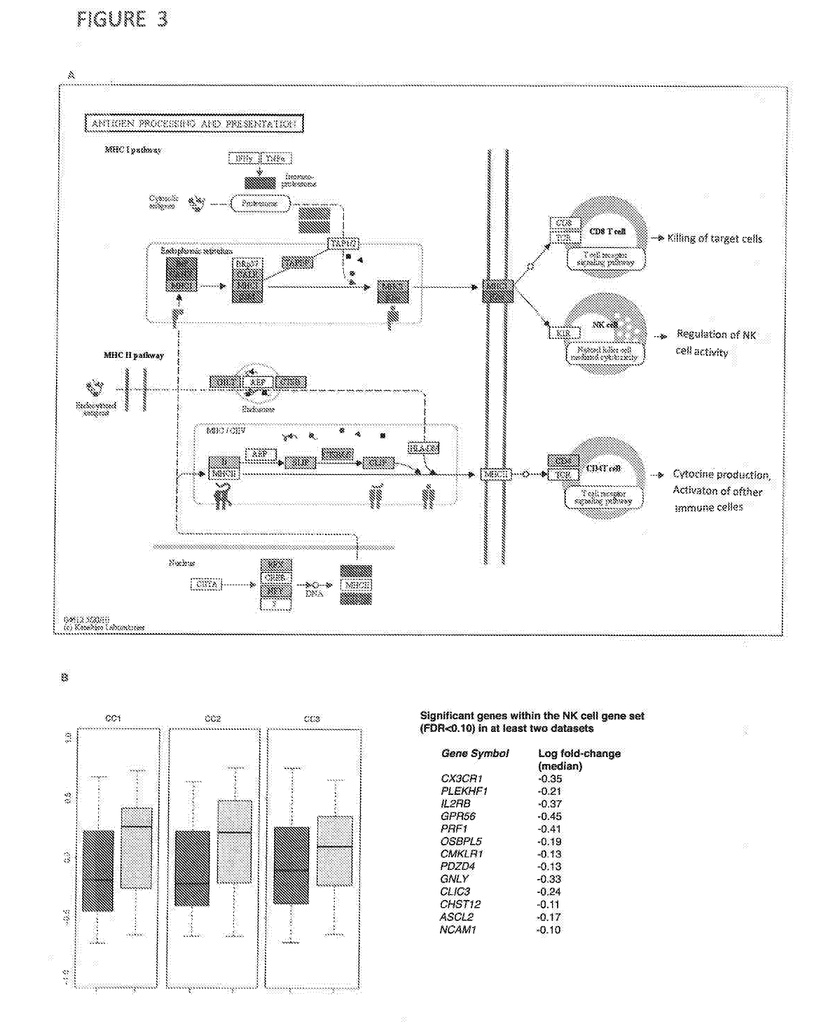

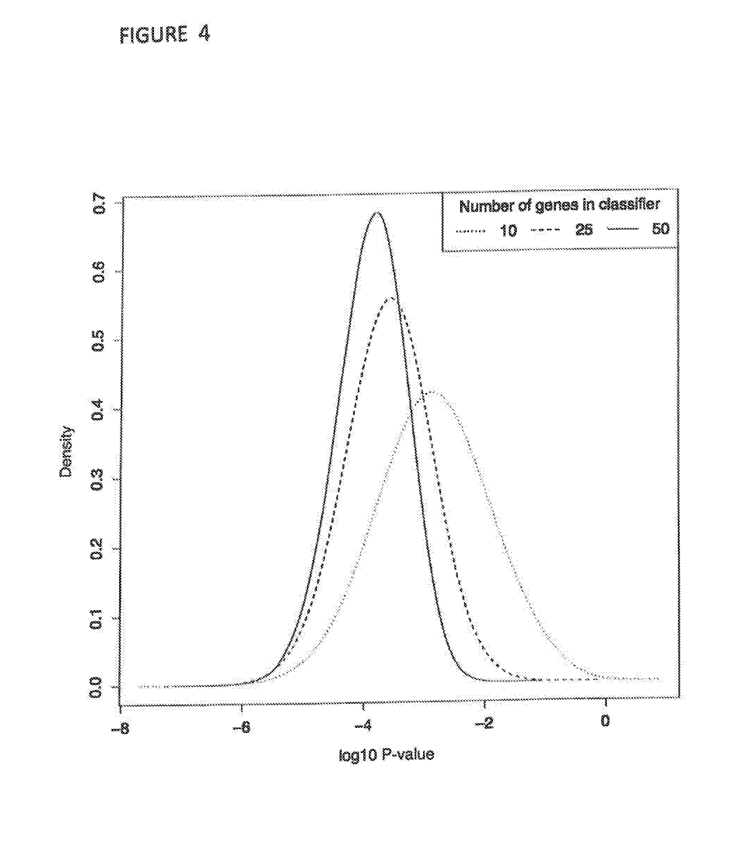

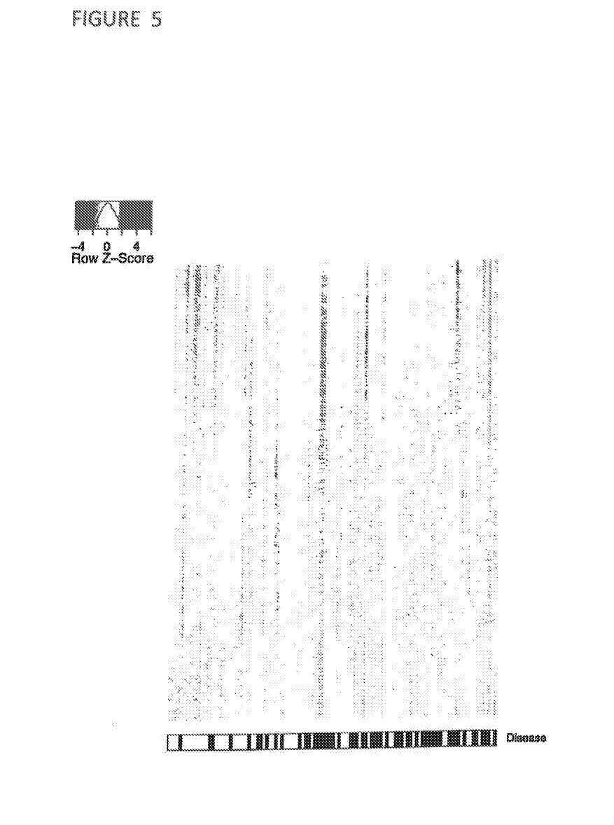

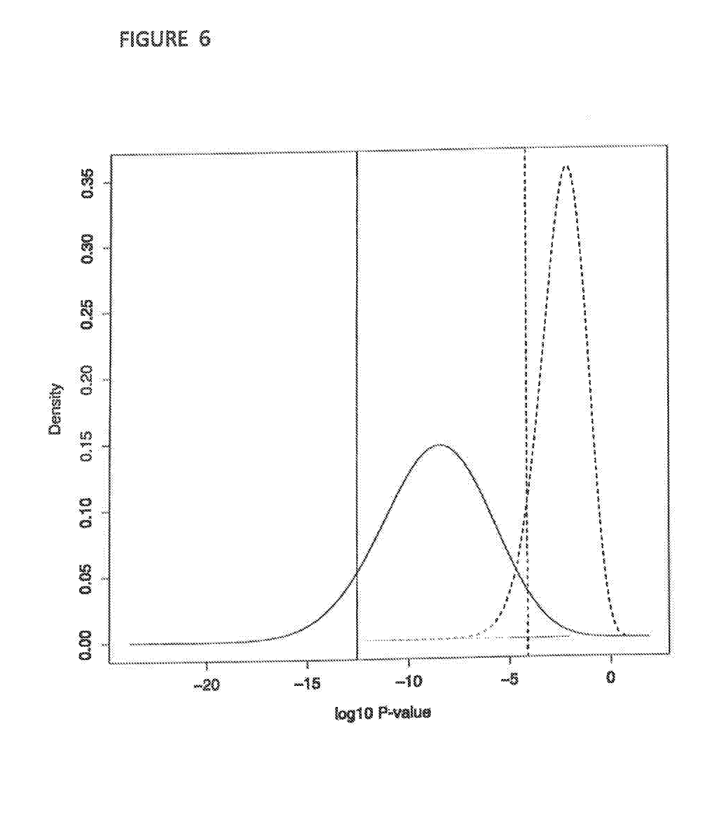

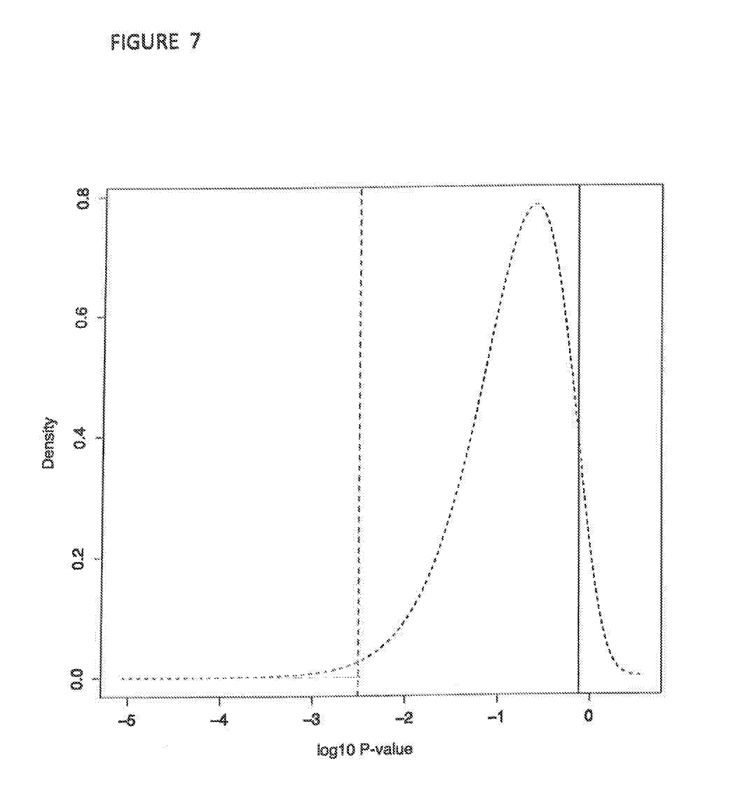

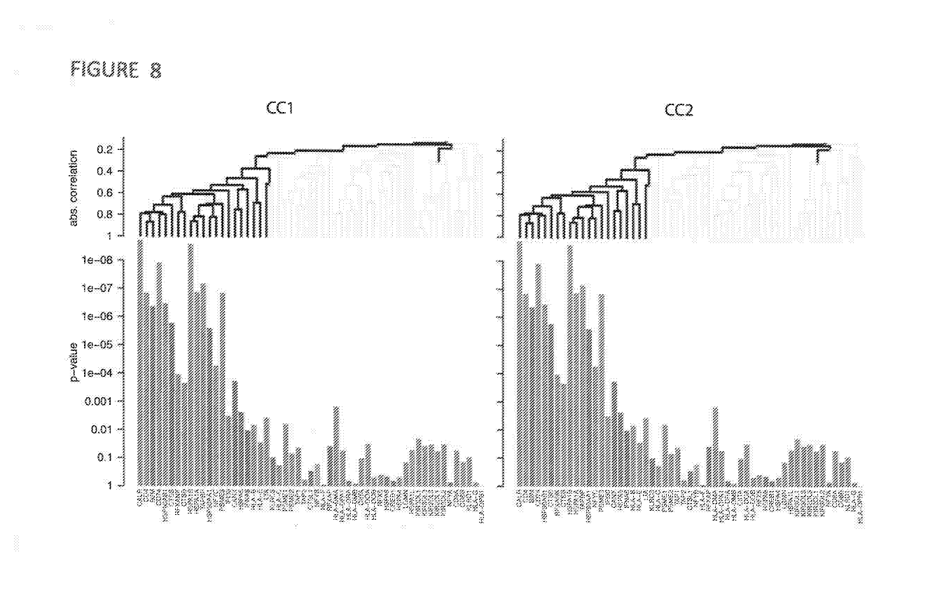

To test whether these findings were replicable in an independent data set (CC2), we investigated blood gene expression profiles from an additional 49 pairs of breast cancer cases and controls subjected to the same data processing as CC1 (Table 3). 418 of the 7898 genes passing quality controls were differentially expressed in CC2 with a FDR<0.005, of which 345 were also differentially expressed in CC1 (P=3×10⁻⁶⁰, hypergeometric test; FIG. 1A, Table 4). Remarkably, the directionality of differential expression between breast cancer cases and controls of all 345 overlapping genes was conserved between datasets (FIG. 1B). When patients were ranked according to the sum of expression over the 345 overlapping genes, the majority of blood samples from breast cancer cases were segregated from controls in both datasets (FIG. 1C).

### Example 3

Using both CC1 and CC2 to select genes differentially expressed in blood samples from breast cancer patients compared to controls, we next asked whether we could accurately classify a third independent dataset encompassing (CC3). Data were subjected to the same processing as CC1 and CC2 (Table 3) and included the expressions of 8529 unique genes across 59 new case-control pairs from NOWAC. Of note, amplified RNA from the blood samples in CC3 was hybridized using a different version of the Illumina array system that includes 12 samples per array with about 40% less probes per signal. That can explain, at least partly, the higher FDR associated with the 345-gene list (Table 4). We built a predictor including all 341 expressed genes in CC3 of the 345-gene list and accurately predicted disease status in this validation dataset (P=8.7×10⁻⁵; fisher test; FIG. 1D). We investigated the distribution of accuracy significances that can be obtained from 100,000 predictors built using 50 genes among the 341 expressed genes, and compared it with predictors using 50 random genes present in CC3 (N=8529, FIG. 1D). The “best” 50-gene predictor chosen among the 341 significant genes (Table 4) had an accuracy of 72.9% to predict the presence of breast cancer (sensitivity=83.1% and specificity=62.7%; P=3.0×10⁻⁹; fisher test). In all three datasets, we investigated whether RNA quality quantified by the RIN value and the use of menopausal hormone therapies or other specific medications could explain the misclassification of controls (i.e. false positives) and cases (i.e. false negatives). In CC2 only, true and false positives had a significantly lower RNA quality defined by the RIN value compared to false and true negatives, respectively (P=1×10⁻³ and 0.02; student test). This indicates that gene expression changes in blood cells of breast cancer patients compared to controls can be confounded by RNA degradation although all samples were selected with a good RNA quality (RIN>7). Also, a significant proportion of controls misclassified as cases in CC3 (36.4%) were currently using of either a selective serotonin reuptake inhibitors (ATC N06AB) or a selective beta-blocking agent (ATC C07AB). Both drugs were previously found to inhibit the expression of T-cell and adaptive immunity-related genes ⁹⁻¹¹ that may explain the lower specificity in CC3 of the best 50-gene predictor identified. Notably, perturbagen signatures from the connectivity map associated with histone deacetylase, Hsp90, tyrosine kinase and immune response inhibitors were significantly enriched in our 50-gene predictor (Table 9, Table 5). Overall, this indicates that our 50-gene predictor contains cytostatic signals including the specific suppression of tumor immunity and that medications influencing transcription involved in those processes can be confounder of the blood-based signal associated with the presence of breast cancer. Considering breast cancer samples included in all 3 datasets, we did not observe tumor receptor specific or stage specific differences between true and false positives.

### Example 4

To further validate the results, we investigated whether we were able to predict breast cancer diagnosis in two external datasets deposited in NCBI's Gene Expression Omnibus¹² including gene expression profiles of peripheral blood mononuclear cells (PBMC) from breast cancer patients, pre- and post-surgery samples, patients from benign breast diseases, controls, gastrointestinal and brain cancer patients¹³ (GSE27562) and gene expression profiles of whole blood cells from breast cancer patients and controls with suspect mammograms¹⁴ (GSE164430). In the PBMC dataset, our 50-gene predictor was able to accurately predict the presence of breast cancer compared to controls from PBMC gene expression profiles (91.5% accuracy, FIG. 6). This indicates that our diagnostic profile for breast cancer identified from whole blood cells is clearly found in circulating mononuclear cells including monocytes, T-cells, B-cells, and natural killer (NK) cells. Of note, the naïve bayes classifier based on the top 3 factors published in LaBreche et al. (N=77 genes) achieved a significantly lower accuracy (79.3%; P=0.02 McNemar χ² test) in their own dataset. All PBMC samples from other cancer types were predicted as normal, which indicates that our predictor is specific for breast cancer. Since our predictor was not trained to differentiate malignant breast cancer from benign breast diseases, we obtained significantly lower accuracy when we included those samples (63.4% accuracy; FIG. 6). The expressions of only 33 genes of our 50-gene predictor were available in the second dataset (GSE164430) although we were able to significantly predict breast cancer diagnosis compared to women with suspect screening mammograms (P=0.008; fisher test, FIG. 7). We were not granted access of the Breast Cancer Test gene list derived from this study. In conclusion, our blood-based gene expression analysis produced uniquely robust and reproducible results across microarray platforms and external datasets to specifically detect breast cancer from population-based controls, warranting further analysis of the processes found deregulated by the presence of a breast cancer in circulating blood cells including PBMC.

### Example 5

### Pathway and Gene Set Variation Analyses

Pathway enrichment analysis showed that our 345-gene list differentially expressed in blood with the presence of breast cancer patients was enriched with a FDR<0.10 for gene ontology categories related to apoptosis, RNA binding, spliceosome and RNA splicing, protein synthesis, RNA metabolism, transcriptional regulation, cell cycle, metabolism and signal transduction (Table 10). Expert functional annotation curated from GeneCards (Table 4) revealed additional grouping of genes involved in immune processes, cell growth/proliferation, cytoskeletal regulation, signal transduction, protein and cell metabolism. To further investigate how breast cancer affects gene expression in blood, we performed gene set variation analysis in CC1 and CC2 datasets and validate the results in CC3. The collection of gene sets used in the analysis consisted of release 3.0 of the C2 (curated gene sets) and C5 (Gene ontology gene sets) sub-collections of the Molecular Signatures Database (http://www.broad.mit.edu/gsea/msigdb/) of size ranging from 10 to 500 genes. We found 58 gene sets overlapping between the top 200 gene sets deregulated in CC1 and CC2 (FDR<2×10⁻⁴; linear analysis). Although we previously identified a confounding factor with the current use of specific drugs by controls in CC3, forty-five of the 58 significant gene sets overlapping in CC1 and CC2 were validated in CC3 (FDR<0.15, Table 6) and showed remarkably comparable enrichment scores according to disease status in all three datasets (FIG. 2). Gene set variation analysis revealed similar processes seen after functional clustering of our 345-gene list including apoptosis, metabolism and transcription pathways, but also identified additional gene signatures notably involved in antigen processing and presentation and Myc target genes (FIG. 2, Table 6).

### Example 6

### Circulating Blood Cells in Breast Cancer Patients Expressed Changes in Genes Related to Immune Functions

### The Antigen Processing and Presentation Pathway and NK-Cell Mediated Immunity

The antigen processing and presentation (APP) pathway downregulated in blood cells of BC patients (FIG. 2, Table 6) was the most direct evidence that our signature is based specifically on the transcriptome of circulating immune effector cells. Mechanisms that regulate APP alter the form and the quantity of the epitopes that are presented by the major histocompatibility complex (MHC) molecules for immune recognition and can dictate tumor immunogenicity^{19,20}.

We investigated the overlapping core genes driving the observed association of the APP pathway with the presence of breast cancer within CC1 and CC2 (FIG. 3A, FIG. 8). All core genes that part of the MHC class II pathway were downregulated in blood samples from breast cancer patients compared to controls including the interferon gamma-inducible protein 30 (IFI30) and cathepsin S (CTSS) involved in the endocytic generation of MHC class II-restricted epitopes as well as CD74 involved in the formation and transport of MHC class II protein and CD4 a co-receptor that assists the T cell receptor (TCR) (FIG. 3A). A recent study integrating measurements of copy number and gene expression in breast cancer tumor tissue identified trans-acting deletion hotspots localized to both TCR loci. Given that genomic copy number loss at the TCR loci derived a trans-acting immune response module, we asked whether cognate mRNAs modulated by both TCR loci in tumor-infiltrating lymphocytes (N=114, Table 7) were differentially expressed in circulating blood cells of breast cancer patients included in CC1 and CC2. The gene set was significantly enriched when comparing the gene expression profiles of circulating blood cells from breast cancer patients compared to controls (P=1.4×10⁻⁸ and 1.5×10⁻⁵ in CC1 and CC2, respectively; global test). Ranking the samples according to the sum of expression over 42 genes defined as core genes in at least one of the dataset, the majority of blood samples from breast cancer cases were segregated from controls in both datasets (FIG. 8). This indicates that part of the signal from tumor-infiltrating lymphocytes with rearranged TCR loci was also found in circulating blood cells of breast cancer patients compared to controls.

Within the MHC class I pathway, PSME3 of the immune-proteasome was defined as a core gene in the APP pathway downregulated in breast cancer patients (FIG. 3A). In addition, three genes coding for the proteasome (PSMB2, PSMB10, PSMD1) within our 345-gene list (Table 5) were downregulated in blood cells of breast cancer patients compared to controls. These findings support that alterations in epitope processing occur in blood cells of breast cancer patients owing at least partly to changes in the proteasomal machinery. Also, core genes involved in peptide-loading onto MHC class I molecules (TAPBP, CALR) were downregulated in breast cancer patients (FIG. 3A). Finally, several heat shock proteins within the APP pathway were also defined as core genes (FIG. 3A) including the downregulation in breast cancer blood samples of HSP70-1 and -2 genes in breast cancer blood samples (HSPA1A and HSPA1B) that encode the major heat-inducible 70 kDa heat shock protein (HSP70). In contrast, we observed upregulation in breast cancer blood samples of HSPA5 with anti-inflammatory properties.

When investigating the expression of CD markers of no more than 3 immune cell subtype²¹ and transcripts specifically overexpressed in each differentiated immune cell subtypes²², genes specific to NK cells (Table 8) were consistently downregulated within the set and in samples from breast cancer patients compared to controls (FIG. 3B). Consistent with this finding, the NK cell-mediated cytotoxicity pathway from KEGG was significantly downregulated in blood samples from breast cancer patients compared to controls (mean FDR=0.08 across all three datasets; gene set variation analysis). Overall, this indicates that breast cancer evasion of anti-tumor immunity is associated with the downregulation of the APP pathway and NK cell-mediated immunity not only in murine models or in the TME of good prognosis solid tumors²³ but also in the transcriptome of circulating blood cells. Remarkably, one epidemiological study has linked low peripheral blood NK cell cytotoxic activity with increased cancer risk²⁴. Changes in expression of several cytoskeleton-regulating genes among our 345-gene list (Table 4) may imply further deregulation of the APP pathway as well as the disruption of the overall immune response against the damaged cells. For example, integrin-linked kinase (ILK), signaling modules of GTPase-mediated actin polymerization (ABR, ABI3, ARHGAP1), several actin-related genes (ACTB, ACTG1, ARPC5L, PFN1), several genes involved in microtubule-based motor proteins dynein and kinesin (DYNLRB1, DYNC1H1, KIF13B), and two engulfment and cell motility genes (ELMO1, ELMO2) were downregulated in blood cells of breast cancer patients (Table 6). A deregulated cellular cytoskeleton could disrupt the vacuolar transport of MHC-peptide complex in blood cells of breast cancer patients, as well as deregulate the cellular programs that lead to activation, proliferation, differentiation, secretion, cell-cell interaction and survival of immune cells, and phagocytosis²⁵. During the activation of a resting lymphocyte, large metabolic demands are also placed on the cell as it initiates proliferation and cytokine production^{26,27}. However, it has been shown that antigen stimulation signaling is required to control the ability of resting cells to take up and utilize nutrients at levels sufficient to maintain viability²⁸. Consistent with the defect in the APP pathway in blood cells from breast cancer patients, we observed a lowered cell metabolism with an overall downregulation of glycolysis and glucose metabolism (Table 10, FIG. 2). In accordance with our results, altered plasma levels of enzymes involved in glucose, lipid, and amino acid metabolism were observed during tumor development in mice²⁹ indicating the presence of a tumor triggers systemic metabolic dysregulation. When extrinsic growth factors are limiting like it is suggested in blood cells of breast cancer patients, cells can activate pathways such as autophagy that promotes the degradation of intracellular constituents to provide a source of ATP. In accordance with this, two genes promoting autophagy (ATG12, VPM1) were upregulated in blood cells of breast cancer patients (Table 5).

If cells begin with lower energetic levels and less biomass, they have to grow more to reach a size sufficient to enter a replicative division ^{28,30}. Protein synthesis has been defined as a key determinant of cell growth and proliferation³¹ and is regulated by either the rates of translation initiation and elongation or ribosome biogenesis³²⁻³⁴. Consistent with this, three components of the eukaryotic initiation factor 4 complex (EIF4A1, EIF4A3, EIF4H) were significantly downregulated in breast cancer patients compared to controls. Furthermore, several genes involved in ribosomal biogenesis (GAR1, SURF6, RRS1) were downregulated while several small (RPS3A, RPS29) and large (RPL4, RPL5, RPL7, RPL11, RPL15, RPL21, RPL41) ribosomal proteins (RPs) were upregulated in blood cells from breast cancer patients compared to controls. The accumulation of RPs can occur due to defects in ribosome assembly caused by an imbalance among RPs or caused by a defect in the assembly process³⁵. Recent findings have demonstrated that components of the translational apparatus are multifunctional and that several individual RPs play a role in regulating cell growth, transformation and death³⁵. Such an accumulation of any of several RPs can interface with the p53 system, leading to cell-cycle arrest or to apoptosis. For example, both RPL5 and RPL11 upregulated in blood cells from breast cancer patients have been found necessary for the accumulation of p53 and the consequent G1 arrest³⁶. Also, RPL11 can bind and sequester c-myc, itself a positive promoter of ribosome synthesis, and particularly of RPL11 synthesis^{36,37}.

Consistent with this, we found a decrease in gene set expression of the targets downregulated by Myc³⁸ (FIG. 2) in blood cells from breast cancer patients. The downregulated Myc targets defined as core genes in all three datasets are involved in global gene regulatory networks with specific influence on cell growth and proliferation (ILK, ARPC4, PP2R4, ERBB2, CEBPA). Since some Myc target genes are regulators of cell growth while others function in cell differentiation and proliferation pathway, Myc is apparently poised at the interface of these processes in circulating blood cells from breast cancer patients compared to controls. The corresponding gene set including targets up regulated by Myc³⁸ was upregulated but not significant in blood samples of breast cancer patients compared to controls (mean FDR=0.34 across all three datasets; gene set variation analysis). During immune cell proliferation, a fine regulation of the cell cycle is required to maintain immune cell homeostasis³⁹. In our 345-gene list, differentially expressed genes in the cell cycle regulation (TERF2, CKAP5, CUL4B, MCM3, HBP1, NUDC, CTCF, TUBB, USP9X, H2AFX) in circulating blood cells of breast cancer patients were mostly involved in the arrest at mitosis checkpoint. In GSVA, the gene set associated with the loss of Nlp from mitotic centrosomes required at the onset of mitosis was downregulated in the blood samples from breast cancer patients compared to controls (FIG. 2, Table 6). Further, the integrin signaling pathway was downregulated in blood samples from breast cancer patients compared to controls. That indicates that integrin-mediated intracellular signals including cellular shape, mobility, and progression through the cell cycle were downregulated by the presence of breast cancer.

Together, these findings show that tumor development is associated with, and may depend on, systemic immunosuppression including the impairment of Ag processing/presentation pathway, and the metabolism, growth, motility, and proliferation of immune cells with a remarkable suppression of NK cell-mediated immunity.

The work leading up to the present invention has received funding from the European Research Council under the European Communit's Seventh Framework Programme (FPT/2007-2013)/ERC grant agreement no. 232997.

The human biological material applied in the study presented in present application has been approved by Regional Committees for Medical and Health Research Ethics in Norway; P REK NORD 27/2004 and P REK NORD 146/2006, and is in accordance with the “Biobankloven” (lov av 21 Feb. 2003 no.12 om biobanker).

### REFERENCES

1 Bissell, M. J. & Radisky, D. Putting tumours in context. Nature reviews. Cancer 1, 46-54, doi:10.1038/35094059 (2001).
2 de Visser, K. E., Eichten, A. & Coussens, L. M. Paradoxical roles of the immune system during cancer development. Nature reviews. Cancer 6, 24-37, doi:10.1038/nrc1782 (2006).
3 Hanahan, D. & Weinberg, R. A. Hallmarks of cancer: the next generation. Cell 144, 646-674, doi:10.1016/j.cell.2011.02.013 (2011).
4 Grivennikov, S. I., Greten, F. R. & Karin, M. Immunity, inflammation, and cancer. Cell 140, 883-899, doi:10.1016/j.cell.2010.01.025 (2010).
5 Ogawa, M. Differentiation and proliferation of hematopoietic stem cells. Blood 81, 2844-2853 (1993).
6 Dumeaux, V. et al. Gene expression analyses in breast cancer epidemiology: the Norwegian Women and Cancer postgenome cohort study. Breast cancer research: BCR 10, R13, doi:10.1186/bcr1859 (2008).
7 Lund, E. & Dumeaux, V. Systems epidemiology in cancer. Cancer epidemiology, biomarkers & prevention: a publication of the American Association for Cancer Research, cosponsored by the American Society of Preventive Oncology 17, 2954-2957, doi:10.1158/1055-9965.EPI-08-0519 (2008).
8 Lund, E. et al. Cohort profile: The Norwegian Women and Cancer Study—NOWAC—Kvinner og kreft. International journal of epidemiology 37, 36-41, doi:10.1093/ije/dym137 (2008).
9 Taler, M. et al. Immunomodulatory effect of selective serotonin reuptake inhibitors (SSRIs) on human T lymphocyte function and gene expression. European neuropsychopharmacology: the journal of the European College of Neuropsychopharmacology 17, 774-780, doi:10.1016/j.euroneuro.2007.03.010 (2007).
10 Sacre, S., Medghalchi, M., Gregory, B., Brennan, F. & Williams, R. Fluoxetine and citalopram exhibit potent antiinflammatory activity in human and murine models of rheumatoid arthritis and inhibit toll-like receptors. Arthritis and rheumatism 62, 683-693, doi:10.1002/art.27304 (2010).
11 Gasser, R. Myocardial ischemia and the immune system: some thoughts and changing views J Clin Basic Cardiol 14, 7 (2011).
12 Barrett, T. et al. NCBI GEO: archive for functional genomics data sets—10 years on. Nucleic acids research 39, D1005-1010, doi:10.1093/nar/gkq1184 (2011).
13 LaBreche, H. G., Nevins, J. R. & Huang, E. Integrating factor analysis and a transgenic mouse model to reveal a peripheral blood predictor of breast tumors. BMC medical genomics 4, 61, doi:10.1186/1755-8794-4-61 (2011).
14 Aaroe, J. et al. Gene expression profiling of peripheral blood cells for early detection of breast cancer. Breast cancer research: BCR 12, R7, doi:10.1186/bcr2472 (2010).
15 Rebhan, M., Chalifa-Caspi, V., Prilusky, J. & Lancet, D. GeneCards: integrating information about genes, proteins and diseases. Trends in genetics: TIG 13, 163 (1997).
16 Anderson, P. Post-transcriptional regulons coordinate the initiation and resolution of inflammation. Nature reviews. Immunology 10, 24-35, doi:10.1038/nri2685 (2010).
17 Keene, J. D. RNA regulons: coordination of post-transcriptional events. Nature reviews. Genetics 8, 533-543, doi:10.1038/nrg2111 (2007).
18 Lynch, K. W. Consequences of regulated pre-mRNA splicing in the immune system. Nature reviews. Immunology 4, 931-940, doi:10.1038/nri1497 (2004).
19 Garrido, C. et al. Alterations of HLA class I expression in human melanoma xenografts in immunodeficient mice occur frequently and are associated with higher tumorigenicity. Cancer immunology, immunotherapy: CII 59, 13-26, doi:10.1007/s00262-009-0716-5 (2010).
20 Neefjes, J., Jongsma, M. L., Paul, P. & Bakke, O. Towards a systems understanding of MHC class I and MHC class II antigen presentation. Nature reviews. Immunology 11, 823-836, doi:10.1038/nri3084 (2011).
21 Curtis, C. et al. The genomic and transcriptomic architecture of 2,000 breast tumours reveals novel subgroups. Nature 486, 346-352, doi:10.1038/nature10983 (2012).
22 Birnbaum, K. D. & Kussell, E. Measuring cell identity in noisy biological systems. Nucleic acids research 39, 9093-9107, doi:10.1093/nar/gkr591 (2011).
23 Watkins, N. A. et al. A HaemAtlas: characterizing gene expression in differentiated human blood cells. Blood 113, el-9, doi:10.1182/blood-2008-06-162958 (2009).
24 Vivier, E., Ugolini, S., Blaise, D., Chabannon, C. & Brossay, L. Targeting natural killer cells and natural killer T cells in cancer. Nature reviews. Immunology 12, 239-252, doi:10.1038/nri3174 (2012).
25 Imai, K., Matsuyama, S., Miyake, S., Suga, K. & Nakachi, K. Natural cytotoxic activity of peripheral-blood lymphocytes and cancer incidence: an 11-year follow-up study of a general population. Lancet 356, 1795-1799, doi: 10.1016/S0140-6736(00)03231-1 (2000).
26 Vicente-Manzanares, M. & Sanchez-Madrid, F. Role of the cytoskeleton during leukocyte responses. Nature reviews. Immunology 4, 110-122, doi:10.1038/nri1268 (2004).
27 Jones, R. G. & Thompson, C. B. Revving the engine: signal transduction fuels T cell activation. Immunity 27, 173-178, doi:10.1016/j.immuni.2007.07.008 (2007).
28 Fox, C. J., Hammerman, P. S. & Thompson, C. B. Fuel feeds function: energy metabolism and the T-cell response. Nature reviews. Immunology 5, 844-852, doi:10.1038/nri1710 (2005).
29 Rathmell, J. C., Vander Heiden, M. G., Harris, M. H., Frauwirth, K. A. & Thompson, C. B. In the absence of extrinsic signals, nutrient utilization by lymphocytes is insufficient to maintain either cell size or viability. Molecular cell 6, 683-692 (2000).
30 Pitteri, S. J. et al. Tumor microenvironment-derived proteins dominate the plasma proteome response during breast cancer induction and progression. Cancer research 71, 5090-5100, doi:10.1158/0008-5472.CAN-11-0568 (2011).
31 Frauwirth, K. A. & Thompson, C. B. Activation and inhibition of lymphocytes by costimulation. The Journal of clinical investigation 109, 295-299, doi:10.1172/JCI14941 (2002).
32 Pardee, A. B. G1 events and regulation of cell proliferation. Science 246, 603-608 (1989).
33 Holland, E. C., Sonenberg, N., Pandolfi, P. P. & Thomas, G. Signaling control of mRNA translation in cancer pathogenesis. Oncogene 23, 3138-3144, doi:10.1038/sj.onc.1207590 (2004).
34 Thomas, G. An encore for ribosome biogenesis in the control of cell proliferation. Nature cell biology 2, E71-72, doi:10.1038/35010581 (2000).
35 Warner, J. R., Vilardell, J. & Sohn, J. H. Economics of ribosome biosynthesis. Cold Spring Harbor symposia on quantitative biology 66, 567-574 (2001).
36 Warner, J. R. & McIntosh, K. B. How common are extraribosomal functions of ribosomal proteins? Molecular cell 34, 3-11, doi:10.1016/j.molcel.2009.03.006 (2009).
37 Dai, M. S., Arnold, H., Sun, X. X., Sears, R. & Lu, H. Inhibition of c-Myc activity by ribosomal protein L11. The EMBO journal 26, 3332-3345, doi:10.1038/sj.emboj.7601776 (2007).
38 van Riggelen, J., Yetil, A. & Felsher, D. W. MYC as a regulator of ribosome biogenesis and protein synthesis. Nature reviews. Cancer 10, 301-309, doi:10.1038/nrc2819 (2010).
39 Zeller, K. I., Jegga, A. G., Aronow, B. J., O'Donnell, K. A. & Dang, C. V. An integrated database of genes responsive to the Myc oncogenic transcription factor: identification of direct genomic targets. Genome biology 4, R69, doi:10.1186/gb-2003-4-10-r69 (2003).
40 Baek, K. H. et al. p53 deficiency and defective mitotic checkpoint in proliferating T lymphocytes increase chromosomal instability through aberrant exit from mitotic arrest. Journal of leukocyte biology 73, 850-861 (2003).
41 Lund, E. et al. External validity in a population-based national prospective study—the Norwegian Women and Cancer Study (NOWAC). Cancer causes & control: CCC 14, 1001-1008 (2003).
42 Smyth, G. K. Linear models and empirical bayes methods for assessing differential expression in microarray experiments. Statistical applications in genetics and molecular biology 3, Article 3, doi:10.2202/1544-6115.1027 (2004).
43 Benjamini, Y. & Hochberg, Y. Controlling the false discovery rate: a practical and powerful approach to multiple testing. Journal of the Royal Statistical Society Series B 57, 289-300 (1995).
44 Huang da, W., Sherman, B. T. & Lempicki, R. A. Systematic and integrative analysis of large gene lists using DAVID bioinformatics resources. Nature protocols 4, 44-57, doi:10.1038/nprot.2008.211 (2009).
45 Ashburner, M. et al. Gene ontology: tool for the unification of biology. The Gene Ontology Consortium. Nature genetics 25, 25-29, doi:10.1038/75556 (2000).
46 Ogata, H. et al. KEGG: Kyoto Encyclopedia of Genes and Genomes. Nucleic acids research 27, 29-34 (1999).
47 Subramanian, A. et al. Gene set enrichment analysis: a knowledge-based approach for interpreting genome-wide expression profiles. Proceedings of the National Academy of Sciences of the United States of America 102, 15545-15550, doi:10.1073/pnas.0506580102 (2005).
48 Goeman, J. J., van de Geer, S. A., de Kort, F. & van Houwelingen, H. C. A global test for groups of genes: testing association with a clinical outcome. Bioinformatics 20, 93-99 (2004).
49 WO 2011/086174 A2
50 Aarøe, J. et al. Gene expression profiling of peripheral blood cells for early detection of breast cancer. Breast Cancer Research 12, no. 1, R7 (2010)

We claim:

1. An in vitro method for diagnosing, identifying or monitoring proliferative disorder in a subject, which method comprises the following step:
a) measuring the level of gene expression in a subset of genes set forth in Table 1 or 2. in a sample from said subject;
b) comparing the level of gene expression of the subset of genes in the sample from said subject with the level of gene expression of the subset of genes in a standard gene expression pattern extracted from healthy subjects;
c) wherein change of gene expression of each and one gene of said subset in said sample as compared to a standard gene expression pattern being indicative for a proliferative disorder.

2. The method of claim 1, wherein the subset of genes are at least about 50, preferably at least about 30, more preferably at least about 20, most preferably at least 10 and even more preferably at least 2.

3. The method of any one of claims 1 or 2, wherein said subset of genes comprises all the genes set forth in Table 1 or 2.

4. The method of any one of claims 1-3, wherein said subset of genes is selected from the genes associated with systemic immunosuppression, cell motility, metabolism and/or proliferation selected from Table 1 or 2.

5. The method of anyone of claims 1 to 4, wherein the change of gene expression is measured to be at least 10%, preferably at least 20%, more preferably at least 30% when measured as a intensity value of scanned images.

6. The method of any one of claims 1 to 5, wherein the level of gene expression is measured using oligonucleotide probes selected from Table 1 or 2 or oligonucleotides derived from a sequence set forth in Table 1 or 2 or any combination thereof, or an oligonucleotide with a complementary sequence, or a functional equivalent oligonucleotide.

7. The method of claim 6, wherein said oligonucleotide probes is a set of probes of at least about 50, preferably at least about 30, more preferably at least about 20, most preferably at least about 10 and most preferably at least about 2 oligonucleotide probes.

8. The method of any one of claims 1 to 7, wherein the oligonucleotide probes hybridize under high stringency conditions with the subset of genes of Table 1 or 2.

9. The method of any one of claims 1 to 8, wherein the oligonucleotide probes are immobilized on one or more solid supports.

10. The method of any one of claims 1 to 9, wherein said solid support is a membrane, plate, or a biochip.

11. The method of any one of claims 1 to 10, wherein said gene expression is measured using oligonucleotide probes of at least about 20, 50, 100 or 200 nucleotides in length.

12. The method of claims 1 to 11, wherein the levels of gene expression in said subset of genes are detected in said sample by determining the levels of RNA molecules encoded by said genes.

13. The method of claims 1 to 12, wherein the level of RNA molecules are detected by using a micro array technique.

14. The method according to any one of claims 1 to 13, wherein the sample is blood cells.

15. The method according to any one of claims 1 to 14, wherein the proliferative disorder is cancer and preferably breast cancer.

16. The method according to any one of claims 1 to 15, wherein the subject is a human.

17. An in vitro method according to claim 1, for preparing a standard gene expression pattern reflecting proliferative disorder in a subject, which method comprises the following step:
a) measuring the level of gene expression in a sample from said subject,
b) measuring level of gene expression in a control sample from a healthy subject;
c) comparing level of gene expression of the sample from said subject with the level of gene expression in a control sample from the healthy subject (not suffering from a proliferative disease) to produce a characteristic standard gene expression pattern reference from genes reflecting proliferative disorder as set forth in Table 1 or 2.

18. A set of oligonucleotide probes, wherein said set is selected from the oligonucleotides of Table 1 or 2 or oligonucleotides derived from a sequence set forth in Table 1 or 2 or any combination thereof, or a oligonucleotide with a complementary sequence, or a functional equivalent oligonucleotide.

19. A set of oligonucleotide probes according to claim 18, wherein said set comprises at least about 50, preferably at least about 30, more preferably at least about 20, most preferably at least about 10 and most preferably at least about 2 oligonucleotide probes.

20. A set of oligonucleotide probes according to claim 19, wherein said set hybridize under high stringency conditions with the subset of genes of Table 1 or 2.

21. A set of oligonucleotide probes according to any one of claims 18 to 20, wherein said probes are immobilized on one or more solid supports.

22. A set of oligonucleotide probes according to claim 21, wherein said solid support is a membrane, plate or biochip.

23. A kit for in vitro diagnosing, identifying or monitoring proliferative disorder comprising:
a collection of oligonucleotide probes and/or primers capable of detecting the level of expression of at least about 50, preferably at least about 30, more preferably at least about 20, most preferably at least 10 and even more preferably at least 2, set forth in Table 1 or 2 or any combination thereof.

24. The kit of claim 23, wherein said probes are capable of specifically hybridizing to RNA transcripts of said genes.

25. A kit comprising a set of oligonucleotide probes as defined in anyone of claims 18-22.

26. Use of a method of any one of claims 1 to 17, or a set of oligonucleotide probes of any one of claims 18-21, or a kit of any one of claims 22 to 25 for diagnosing, identifying or monitoring proliferative disorder in a subject.


SEQUENCE LISTING (545 sequences)

SEQ ID NO: 1 - DNA, 50 bp, Artificial Sequence
  Oligonucleotide
gtgctctttgttcatcattggccctcattccaagcactttacgctgtctg

SEQ ID NO: 2 - DNA, 50 bp, Artificial Sequence
  Oligonucleotide
ccaggcagagactacctttgtgaccagctcccagtaaaaaccccaggcac

SEQ ID NO: 3 - DNA, 50 bp, Artificial Sequence
  Oligonucleotide
tgtcctgtgactgccccacagagataaggggccaggagggattgaaaggc

SEQ ID NO: 4 - DNA, 50 bp, Artificial Sequence
  Oligonucleotide
aaacgccaggtctgcctgttcttgctgggcaatggctgatggctgccagt

SEQ ID NO: 5 - DNA, 50 bp, Artificial Sequence
  Oligonucleotide
taaggagaatggcccagtcctctcccaagtccacacaggggaggtgatag

SEQ ID NO: 6 - DNA, 50 bp, Artificial Sequence
  Oligonucleotide
cggctacagcttcaccaccacggccgagcgggaaatcgtgcgtgacatta

SEQ ID NO: 7 - DNA, 50 bp, Artificial Sequence
  Oligonucleotide
acaggaagtcccttgccatcctaaaagccaccccacttctctctaaggag

SEQ ID NO: 8 - DNA, 50 bp, Artificial Sequence
  Oligonucleotide
gaggctggcaagaaccagttgttttgtcttgcgggtctgtcagggttgga

SEQ ID NO: 9 - DNA, 50 bp, Artificial Sequence
  Oligonucleotide
cgtttctctgccggtcgcaatggaagaagagatcgccgcgctggtcattg

SEQ ID NO: 10 - DNA, 50 bp, Artificial Sequence
  Oligonucleotide
agctcctttgtcgctctcatggctgtcagatcctggtccctccacactgg

SEQ ID NO: 11 - DNA, 50 bp, Artificial Sequence
  Oligonucleotide
gtggaattggcaaacccactgcagcccctgagaggaggtcgaatgggtaa

SEQ ID NO: 12 - DNA, 50 bp, Artificial Sequence
  Oligonucleotide
agttccacaggctcccgtccagggggaggactacggcaaaggtgtcatct

SEQ ID NO: 13 - DNA, 50 bp, Artificial Sequence
  Oligonucleotide
cagtgtgacagtgccagccaatgtgcagaggtggatgaggtcttgtgaaa

SEQ ID NO: 14 - DNA, 50 bp, Artificial Sequence
  Oligonucleotide
gcaggcaggaacagtgtgggtgaattgctatggcgtggtaagtgcccagt

SEQ ID NO: 15 - DNA, 50 bp, Artificial Sequence
  Oligonucleotide
caattctcccaatgagccttttgtctgtgggaaaagcaggagacgcttcg

SEQ ID NO: 16 - DNA, 50 bp, Artificial Sequence
  Oligonucleotide
ctacctgcttggcctggggcctctggaagtctgctgtgaatttgtattcc

SEQ ID NO: 17 - DNA, 50 bp, Artificial Sequence
  Oligonucleotide
ctctcaggggccagaactcctttgccagcgtggatttctcaagtcgggac

SEQ ID NO: 18 - DNA, 50 bp, Artificial Sequence
  Oligonucleotide
ggtgtgaacctacctgccttggagagggcccaggtcccaaatctcttcaa

SEQ ID NO: 19 - DNA, 50 bp, Artificial Sequence
  Oligonucleotide
ggttcctgtagcagcagccgaagggagacctcgcttaaaccaacacaatc

SEQ ID NO: 20 - DNA, 50 bp, Artificial Sequence
  Oligonucleotide
cctggtggctctttgtggaggaaactaaacattcccttgatggtctcaag

SEQ ID NO: 21 - DNA, 50 bp, Artificial Sequence
  Oligonucleotide
cgtgcttggggttcagctggtgaggctgtccctgtaggaagaaagctctg

SEQ ID NO: 22 - DNA, 50 bp, Artificial Sequence
  Oligonucleotide
tgtggtggagatgactgaagcccgacacggcctgagcgtccagaaatggt

SEQ ID NO: 23 - DNA, 50 bp, Artificial Sequence
  Oligonucleotide
gtgcctgcaccaccccactgccttccttcagcacctttagctgcatttgt

SEQ ID NO: 24 - DNA, 50 bp, Artificial Sequence
  Oligonucleotide
gccttctctgcctgtccctccgatccttgtccaccgtctatttattgccc

SEQ ID NO: 25 - DNA, 50 bp, Artificial Sequence
  Oligonucleotide
ttatacggatgactgggaggcactgcaccacaacgtaggaccctggctcc

SEQ ID NO: 26 - DNA, 50 bp, Artificial Sequence
  Oligonucleotide
cctagctccttgttggtgagcttcttgtgccttaatcctgtgacccagcc

SEQ ID NO: 27 - DNA, 50 bp, Artificial Sequence
  Oligonucleotide
acccccacagaccccgttcctccagcctgcgtgcccctaacctggctttt

SEQ ID NO: 28 - DNA, 50 bp, Artificial Sequence
  Oligonucleotide
gcagagctgtgcttcctggacgtgattcccttttggaagctggacctgga

SEQ ID NO: 29 - DNA, 50 bp, Artificial Sequence
  Oligonucleotide
catcatgctgggggagattctcagacactcgatggatccacccacattca

SEQ ID NO: 30 - DNA, 50 bp, Artificial Sequence
  Oligonucleotide
atgatggcggtggaggtggtggttgtagtgtgatggatcccctttaggtt

SEQ ID NO: 31 - DNA, 50 bp, Artificial Sequence
  Oligonucleotide
tcattggactcatggtgggcggtgttgtcatagcgacagtgatcgtcatc

SEQ ID NO: 32 - DNA, 50 bp, Artificial Sequence
  Oligonucleotide
gcagaactagacccccgccacagcagcctctgaagttggacagcaaaacc

SEQ ID NO: 33 - DNA, 50 bp, Artificial Sequence
  Oligonucleotide
gagttacaagcaccaggggatgctctacatcaagggatgcaccttcagtc

SEQ ID NO: 34 - DNA, 50 bp, Artificial Sequence
  Oligonucleotide
gctggttgaaaagtaccactcccactctgaacatctggccgtccctgcaa

SEQ ID NO: 35 - DNA, 50 bp, Artificial Sequence
  Oligonucleotide
agggaccaatctggggctggaaatgttaggaggttgccttggtgctgccc

SEQ ID NO: 36 - DNA, 50 bp, Artificial Sequence
  Oligonucleotide
tgggacagtgtggtggtaccaggaagaaagaggattggaaaggccagtac

SEQ ID NO: 37 - DNA, 50 bp, Artificial Sequence
  Oligonucleotide
cctaacaagaattaagcagagttgggggaagtgggaggggtgacaagcat

SEQ ID NO: 38 - DNA, 50 bp, Artificial Sequence
  Oligonucleotide
tctgctctccgccctcctttgtgtatcaagtgtcgctcacagccccattc

SEQ ID NO: 39 - DNA, 50 bp, Artificial Sequence
  Oligonucleotide
gtgactgctggctctgtcacctcatcaaactggatgtgacccatgccgcc

SEQ ID NO: 40 - DNA, 50 bp, Artificial Sequence
  Oligonucleotide
ggagcaggggcaggcgaacctctttctttgcagaccgaacagtgaaaagc

SEQ ID NO: 41 - DNA, 50 bp, Artificial Sequence
  Oligonucleotide
caggccttccctgacccagccaggaacaaacaagggaccaagtgcacaca

SEQ ID NO: 42 - DNA, 50 bp, Artificial Sequence
  Oligonucleotide
actgcagaactgacattttgacggtctaccagcgtggcggctggtgttgg

SEQ ID NO: 43 - DNA, 50 bp, Artificial Sequence
  Oligonucleotide
ttgctccgggacgatgagagtatttcccatgctggagtcggcgagaaggg

SEQ ID NO: 44 - DNA, 50 bp, Artificial Sequence
  Oligonucleotide
cagtgtttcgtgaaggtgttggagaggggctgtgtctgggtgagggatgg

SEQ ID NO: 45 - DNA, 50 bp, Artificial Sequence
  Oligonucleotide
gagtcgtgattgtaccactgcattcctgctgagcaacagagtgagacccc

SEQ ID NO: 46 - DNA, 50 bp, Artificial Sequence
  Oligonucleotide
cgaagtcagaaaactcatcatcaggcgacgccctggcggctgggtggaga

SEQ ID NO: 47 - DNA, 50 bp, Artificial Sequence
  Oligonucleotide
ggagtttggaactctaccctggtaggaaagcaccgcagcatgtggggaag

SEQ ID NO: 48 - DNA, 50 bp, Artificial Sequence
  Oligonucleotide
ccctggcttatgagcaggctgagagtgacatcatgaagagacagcccaga

SEQ ID NO: 49 - DNA, 50 bp, Artificial Sequence
  Oligonucleotide
cccaaccctgctgttaggcctgctgttccctttgctcttgattaggagag

SEQ ID NO: 50 - DNA, 50 bp, Artificial Sequence
  Oligonucleotide
gcctcttcctctgaatagaccagacgccctttcacttagttcagtgccag

SEQ ID NO: 51 - DNA, 50 bp, Artificial Sequence
  Oligonucleotide
gcattcatcgtgaggggtctttgtcctctgtactgtctctctccttgccc

SEQ ID NO: 52 - DNA, 50 bp, Artificial Sequence
  Oligonucleotide
cagcttgtctcttgcctgccactgtgtgaatcggcgacggagcactgcac

SEQ ID NO: 53 - DNA, 50 bp, Artificial Sequence
  Oligonucleotide
ggtccagagtacttgttttcccgatgtgtccagccagctccgcagcagct

SEQ ID NO: 54 - DNA, 50 bp, Artificial Sequence
  Oligonucleotide
cctgcctctgtttttccctcgagaccagccaactctcacatttcagtccg

SEQ ID NO: 55 - DNA, 50 bp, Artificial Sequence
  Oligonucleotide
tgtcttcacggcagcgttttgctcacacagcagcttttgcacgccccagg

SEQ ID NO: 56 - DNA, 50 bp, Artificial Sequence
  Oligonucleotide
gccttaggcttcgatggttcttccaacccccttaatatggcttagggtgg

SEQ ID NO: 57 - DNA, 50 bp, Artificial Sequence
  Oligonucleotide
ggcctgaagatgggagattcctaagtggaggagaactgtgccttactgac

SEQ ID NO: 58 - DNA, 50 bp, Artificial Sequence
  Oligonucleotide
gtactgctttcagtgtgttccccctcagcccctccggcgtgtcaggcata

SEQ ID NO: 59 - DNA, 50 bp, Artificial Sequence
  Oligonucleotide
cagagctggcatttgcacaaacacggcaacactgggtggcatccaagtct

SEQ ID NO: 60 - DNA, 50 bp, Artificial Sequence
  Oligonucleotide
acgtcatagctccttagttctgctcctgtcgccctaacttggcatgggca

SEQ ID NO: 61 - DNA, 50 bp, Artificial Sequence
  Oligonucleotide
ccctcctgccctctgtccattcttagagcataccttcatccactccatgc

SEQ ID NO: 62 - DNA, 50 bp, Artificial Sequence
  Oligonucleotide
ccaaacaccaagtgttagctgccttaaggtttatccggggcattggtggc

SEQ ID NO: 63 - DNA, 50 bp, Artificial Sequence
  Oligonucleotide
acctggtcagcctaaatcttcccagtcccgctgtggagctgtcagtcacc

SEQ ID NO: 64 - DNA, 50 bp, Artificial Sequence
  Oligonucleotide
aaagaagtggagcgtgtcctgaaggagttccaccaggccgggaagcccat

SEQ ID NO: 65 - DNA, 50 bp, Artificial Sequence
  Oligonucleotide
cagccacatttcagacctgctgagactgctgagtgaggaatggcagtgag

SEQ ID NO: 66 - DNA, 50 bp, Artificial Sequence
  Oligonucleotide
gaccagaaaaacaaggtggtcacgaccccagccttcatgtgcgagacggc

SEQ ID NO: 67 - DNA, 50 bp, Artificial Sequence
  Oligonucleotide
gcccctttagtccccgctctggaaggccaggcagtttaggtgtaaatagg

SEQ ID NO: 68 - DNA, 50 bp, Artificial Sequence
  Oligonucleotide
accaagatcgcaccactgcattccagcctaagcaatagagcgagactccc

SEQ ID NO: 69 - DNA, 50 bp, Artificial Sequence
  Oligonucleotide
gtggatggcagttttctgtagttttggggactgtggtagctcttggattg

SEQ ID NO: 70 - DNA, 50 bp, Artificial Sequence
  Oligonucleotide
ggcagcacttatgctctgtgacagtattgtgtgtcatagttgagcagtag

SEQ ID NO: 71 - DNA, 50 bp, Artificial Sequence
  Oligonucleotide
cttcttggccaggggaaaggaccacaaggcaatctggggtgtggacagac

SEQ ID NO: 72 - DNA, 50 bp, Artificial Sequence
  Oligonucleotide
tgctgtctgtatcccttggagtaagaaggtagtggcatgggtggagtgtg

SEQ ID NO: 73 - DNA, 50 bp, Artificial Sequence
  Oligonucleotide
ggtctcagccagccctagagactgcttcttgtgtttgtgtcattctgtcc

SEQ ID NO: 74 - DNA, 50 bp, Artificial Sequence
  Oligonucleotide
tcccctgtacctgtgccaagcctagcacttgtgatgcctccatgccccga

SEQ ID NO: 75 - DNA, 50 bp, Artificial Sequence
  Oligonucleotide
aggagtggctgcagctgactatgtattcctgaactggagccccagacccg

SEQ ID NO: 76 - DNA, 50 bp, Artificial Sequence
  Oligonucleotide
acttgagtggaatcctttcctcacgtactcccacagacgtctgggcctgg

SEQ ID NO: 77 - DNA, 50 bp, Artificial Sequence
  Oligonucleotide
actccaagggccaaagctcaaatgcccaccatagaacgactgtccatgac

SEQ ID NO: 78 - DNA, 50 bp, Artificial Sequence
  Oligonucleotide
aaggatctgtgttagtccctgggatggctccaaggcctgctctaggaagg

SEQ ID NO: 79 - DNA, 50 bp, Artificial Sequence
  Oligonucleotide
tgacttgagtccacaaggacacaaacacctgagtagctgggcagcccttg

SEQ ID NO: 80 - DNA, 50 bp, Artificial Sequence
  Oligonucleotide
gggagcagagcttttccctagcacccactttcccaaaccagtctctgcag

SEQ ID NO: 81 - DNA, 50 bp, Artificial Sequence
  Oligonucleotide
agggcggcaccgatcaccgagcagccgtgcgtgtatctcaaggaactaaa

SEQ ID NO: 82 - DNA, 50 bp, Artificial Sequence
  Oligonucleotide
agatgtcctcagacgggaaggtttgagaagggtcagatggtaggcgggcc

SEQ ID NO: 83 - DNA, 50 bp, Artificial Sequence
  Oligonucleotide
aagctttcgtgtgggagccagctatggtgcggatcaatgcgctgacagca

SEQ ID NO: 84 - DNA, 50 bp, Artificial Sequence
  Oligonucleotide
gcccaggggggtacatggtatggagtagacatcaacaacgaggacattgc

SEQ ID NO: 85 - DNA, 50 bp, Artificial Sequence
  Oligonucleotide
aaagtggagcagaggaagctgctggaaaagtgtgccatgaccgctctgag

SEQ ID NO: 86 - DNA, 50 bp, Artificial Sequence
  Oligonucleotide
agagtcctggggagcttctgtccacctgtcctgcagaggagtcgtttcca

SEQ ID NO: 87 - DNA, 50 bp, Artificial Sequence
  Oligonucleotide
cactgctgtacccagatgcctacaaccatccctgccacatacaggtgctc

SEQ ID NO: 88 - DNA, 50 bp, Artificial Sequence
  Oligonucleotide
gttcccagcagggggagaaacccttcacaccccaggcccttcaggaactg

SEQ ID NO: 89 - DNA, 50 bp, Artificial Sequence
  Oligonucleotide
ccccgttcctgacatcacagcagcctccaacacaaggctccaagacctag

SEQ ID NO: 90 - DNA, 50 bp, Artificial Sequence
  Oligonucleotide
tatgggagcatcggctactgctggtgtgtcttccccaacggcacggaggt

SEQ ID NO: 91 - DNA, 50 bp, Artificial Sequence
  Oligonucleotide
aaggcacagggagaagggataaccctacacccagaccccaggctggacat

SEQ ID NO: 92 - DNA, 50 bp, Artificial Sequence
  Oligonucleotide
caggacgagctactgctttggagcgagggtttcctgcttttgagttgacc

SEQ ID NO: 93 - DNA, 50 bp, Artificial Sequence
  Oligonucleotide
aagaggctgcggtgagccaatttagagcccaaagagccccgagggaacct

SEQ ID NO: 94 - DNA, 50 bp, Artificial Sequence
  Oligonucleotide
cactgtctcagagaggttttcctgtgctcgccctgtttctctcaggaagc

SEQ ID NO: 95 - DNA, 50 bp, Artificial Sequence
  Oligonucleotide
actctgcttctctctgtcagcgtcctgctgctctagaagactgtccgtgg

SEQ ID NO: 96 - DNA, 50 bp, Artificial Sequence
  Oligonucleotide
gtacaggcttttagcaccgaagtgtggtcctcagaccagtgcctgccaac

SEQ ID NO: 97 - DNA, 50 bp, Artificial Sequence
  Oligonucleotide
cacgtgtgaacatctgtcttggtcacagagctgggtgctgccggtcacct

SEQ ID NO: 98 - DNA, 50 bp, Artificial Sequence
  Oligonucleotide
gaccctgactgctagttctgaggacactggtggctgtgctatgtgtggcc

SEQ ID NO: 99 - DNA, 50 bp, Artificial Sequence
  Oligonucleotide
gccccgctggtgcactgaagagccaccctgtggaaacactacatctgcaa

SEQ ID NO: 100 - DNA, 50 bp, Artificial Sequence
  Oligonucleotide
tgggaacgggtggcccggctgtgtgactttaaccccaagtctagcaagca

SEQ ID NO: 101 - DNA, 50 bp, Artificial Sequence
  Oligonucleotide
gaggaaagaaattagggcctcctctgatctctcgctatctgcgggtcctg

SEQ ID NO: 102 - DNA, 50 bp, Artificial Sequence
  Oligonucleotide
tgacgcggatggtggggaagaacgtgaagctgtacgacatggtgctgcag

SEQ ID NO: 103 - DNA, 50 bp, Artificial Sequence
  Oligonucleotide
ccagggcagagcctctccttgtactttggcagccatagaaagcgtgctca

SEQ ID NO: 104 - DNA, 50 bp, Artificial Sequence
  Oligonucleotide
aaagcgaggcacactgcttactgccttggggttgtggagatggacccgtg

SEQ ID NO: 105 - DNA, 50 bp, Artificial Sequence
  Oligonucleotide
acgtgctcctgctgaccttctgcggctccgggctgtgtcctaaatgcaaa

SEQ ID NO: 106 - DNA, 50 bp, Artificial Sequence
  Oligonucleotide
ggaaacctgcttctcctactccggttattgtggcctcccacacagccaac

SEQ ID NO: 107 - DNA, 50 bp, Artificial Sequence
  Oligonucleotide
tgcccctctgtctgctgaaggacctgttgctgcttctgtcttttcacccc

SEQ ID NO: 108 - DNA, 50 bp, Artificial Sequence
  Oligonucleotide
ctcccagttctagagcaatctacagctgtttatgtgaggtgcccaacacc

SEQ ID NO: 109 - DNA, 50 bp, Artificial Sequence
  Oligonucleotide
cacagaggtgttctgacctgcaacttgactgggaagcccctgggtaacac

SEQ ID NO: 110 - DNA, 50 bp, Artificial Sequence
  Oligonucleotide
gcagaattgcttgaacccaggaggcagagggttgcagtgagccgagatag

SEQ ID NO: 111 - DNA, 50 bp, Artificial Sequence
  Oligonucleotide
tgttaggttataatttctcatttggagccgggcgcagtggctcacgcctg

SEQ ID NO: 112 - DNA, 50 bp, Artificial Sequence
  Oligonucleotide
gtccctacaggaggaacagtggccttgcttcttagacggtcttcactgtg

SEQ ID NO: 113 - DNA, 50 bp, Artificial Sequence
  Oligonucleotide
aagatacactcctgctgtgcccccatctttcctccaaactcctgcctgtg

SEQ ID NO: 114 - DNA, 50 bp, Artificial Sequence
  Oligonucleotide
cctcaggcagaggatgttctggacctccccctcttggtccctactagaga

SEQ ID NO: 115 - DNA, 50 bp, Artificial Sequence
  Oligonucleotide
cgtgtctcctcggtcgccccgtgtttgcgcttgaccatgttgcactgttt

SEQ ID NO: 116 - DNA, 50 bp, Artificial Sequence
  Oligonucleotide
cctaacccttgaatgactcaaatcagtgccaggtggaggactcccatcac

SEQ ID NO: 117 - DNA, 50 bp, Artificial Sequence
  Oligonucleotide
gctcgagacaattaaggacgtgggatgaggctccgagacaggacgcggtt

SEQ ID NO: 118 - DNA, 50 bp, Artificial Sequence
  Oligonucleotide
aaaccgtcaagcccgaggcggatagagaccacgccagtgaccagttgtag

SEQ ID NO: 119 - DNA, 50 bp, Artificial Sequence
  Oligonucleotide
gaagacggcatcacgaagcagctccaaaaggaaaagcttgggcggtgccc

SEQ ID NO: 120 - DNA, 50 bp, Artificial Sequence
  Oligonucleotide
atgtagcagaatggcacccagaccactgcccaccagtgacggacatgcac

SEQ ID NO: 121 - DNA, 50 bp, Artificial Sequence
  Oligonucleotide
ctgcaggggtcctctgtgaacttgctcaggacaaggaagctgcagaagct

SEQ ID NO: 122 - DNA, 50 bp, Artificial Sequence
  Oligonucleotide
agctgcaggggtcctctgtgaacttgctcaggacaaggaagctgcagaag

SEQ ID NO: 123 - DNA, 50 bp, Artificial Sequence
  Oligonucleotide
cctggatgcggggctctttgttctccagcacatctgctacatcatggccg

SEQ ID NO: 124 - DNA, 50 bp, Artificial Sequence
  Oligonucleotide
gacacttgcacagcatggctctgcctcacaatgatgcagtcagccacctg

SEQ ID NO: 125 - DNA, 50 bp, Artificial Sequence
  Oligonucleotide
gttaggaggagactcttgatgtcaccttcagtatcttgaaagcgggtccc

SEQ ID NO: 126 - DNA, 50 bp, Artificial Sequence
  Oligonucleotide
tgtctgtcatccagctcctatgtctgttatccagctccaagtacagcttg

SEQ ID NO: 127 - DNA, 50 bp, Artificial Sequence
  Oligonucleotide
attgtcattcctgtattcacccgtccagaccttgttcacactctcacatg

SEQ ID NO: 128 - DNA, 50 bp, Artificial Sequence
  Oligonucleotide
ccatctgggaaaataccccatcattcatgctactgccaacctggggagcc

SEQ ID NO: 129 - DNA, 50 bp, Artificial Sequence
  Oligonucleotide
gggcacgcgctgcactccgtaactcaacatggcatgcctttctctccgta

SEQ ID NO: 130 - DNA, 50 bp, Artificial Sequence
  Oligonucleotide
taggttttgtggcatccacggtcaggtgtagaggaagctgccccttgcag

SEQ ID NO: 131 - DNA, 50 bp, Artificial Sequence
  Oligonucleotide
gtcaccatccacgaagctgctgtctctggcacatctccacaattaactcc

SEQ ID NO: 132 - DNA, 50 bp, Artificial Sequence
  Oligonucleotide
ggaaagccagctccccttctcctctaactattggaacgccagaaagtcag

SEQ ID NO: 133 - DNA, 50 bp, Artificial Sequence
  Oligonucleotide
cttccaaagacccactagaatgtcagctgtactctgtactctccactgag

SEQ ID NO: 134 - DNA, 50 bp, Artificial Sequence
  Oligonucleotide
tacctgccctcctactaccacctgcatgtgcacttcaccgccctgggctt

SEQ ID NO: 135 - DNA, 50 bp, Artificial Sequence
  Oligonucleotide
gacagcttactgcagcactgttggtgttcggagctcttctgtgccctggc

SEQ ID NO: 136 - DNA, 50 bp, Artificial Sequence
  Oligonucleotide
accaagggagaaccaggaaacggaaacagagtggtcattccccagcccgg

SEQ ID NO: 137 - DNA, 50 bp, Artificial Sequence
  Oligonucleotide
gttcagggcacaggccccgacatcaccccaaggacaacggcacaagtaga

SEQ ID NO: 138 - DNA, 50 bp, Artificial Sequence
  Oligonucleotide
agtccaggttcaagactagcctgggcaacatggcaagaccctgtccctat

SEQ ID NO: 139 - DNA, 50 bp, Artificial Sequence
  Oligonucleotide
gtctgttgctgctgaaagatgtctgtgtgcctgtatcaacatgtgacttc

SEQ ID NO: 140 - DNA, 50 bp, Artificial Sequence
  Oligonucleotide
aagcaaaaggatggcttgagcccaagagttcagagcagcctggccaccat

SEQ ID NO: 141 - DNA, 50 bp, Artificial Sequence
  Oligonucleotide
ctcctggcttcagtccttacaatgtcagtaaaacagccttgctgggcctc

SEQ ID NO: 142 - DNA, 50 bp, Artificial Sequence
  Oligonucleotide
cagccccattccagggtcccatctgtccggagttgtgtatgtcaagtccg

SEQ ID NO: 143 - DNA, 50 bp, Artificial Sequence
  Oligonucleotide
agcgcctccacctggcctactctgttatttccacctgtttgggtagggcc

SEQ ID NO: 144 - DNA, 50 bp, Artificial Sequence
  Oligonucleotide
gcagcagaggacccaggaccacagtgacacacgaaaggaaacaggctaag

SEQ ID NO: 145 - DNA, 50 bp, Artificial Sequence
  Oligonucleotide
catttctgtaaggcaatcttggcacacgtggggcttaccagtggcccagg

SEQ ID NO: 146 - DNA, 50 bp, Artificial Sequence
  Oligonucleotide
gctacaagtctatcttctttcttgacccatttcaggagggagccctctcc

SEQ ID NO: 147 - DNA, 50 bp, Artificial Sequence
  Oligonucleotide
gccagggttggagccgacgaccagaaaattgcagcaggcactttaaggca

SEQ ID NO: 148 - DNA, 50 bp, Artificial Sequence
  Oligonucleotide
aattcccaggtgttgtggtaggtaattgaatcatgggggcagtttccctc

SEQ ID NO: 149 - DNA, 50 bp, Artificial Sequence
  Oligonucleotide
ctaaaggctacgtcttccagatggagatgattgttcgggcaagacagttg

SEQ ID NO: 150 - DNA, 50 bp, Artificial Sequence
  Oligonucleotide
agccgtggggccaattttttaactcagatcttgctgagaccaggagcatc

SEQ ID NO: 151 - DNA, 50 bp, Artificial Sequence
  Oligonucleotide
ggacgaagaagattacgactcctagcgccttctgccccccagaccatagc

SEQ ID NO: 152 - DNA, 50 bp, Artificial Sequence
  Oligonucleotide
aatatgactattctaaaggctgtgaggccatggggtattggttaagttgc

SEQ ID NO: 153 - DNA, 50 bp, Artificial Sequence
  Oligonucleotide
gcgctgggtcaagcagacaaacaccgagaagaaggccagtgtggtaacct

SEQ ID NO: 154 - DNA, 50 bp, Artificial Sequence
  Oligonucleotide
agccttagtccaggggtgtggctctgtccgggtgcagtatgcagtcatgt

SEQ ID NO: 155 - DNA, 50 bp, Artificial Sequence
  Oligonucleotide
catggcagtcgcttggaacccactcacaccaatccagtgaccgtgtgtgg

SEQ ID NO: 156 - DNA, 50 bp, Artificial Sequence
  Oligonucleotide
agaaaggcacaggactcgctaagtgttcgctacgcggggctaccggatcg

SEQ ID NO: 157 - DNA, 50 bp, Artificial Sequence
  Oligonucleotide
atggcagaggtggaggagacactgaagcgactgcagagccagaagggagt

SEQ ID NO: 158 - DNA, 50 bp, Artificial Sequence
  Oligonucleotide
cattcggtggccgagagcctcaactacgtggcgtcctggaacatgagcat

SEQ ID NO: 159 - DNA, 50 bp, Artificial Sequence
  Oligonucleotide
agcaggtcagaggctcctaactgggcaactcaagattctggcttctactg

SEQ ID NO: 160 - DNA, 50 bp, Artificial Sequence
  Oligonucleotide
cagcagatcagtgggatgagggagactgttcacctgctgtgtactcctgt

SEQ ID NO: 161 - DNA, 50 bp, Artificial Sequence
  Oligonucleotide
aagtggctccgtgaagcagaggaggagtctgaccacaactgagggctggt

SEQ ID NO: 162 - DNA, 50 bp, Artificial Sequence
  Oligonucleotide
cggtggcagtgggtgcctgtagtgtgatgtgtctgaactaataaagtggc

SEQ ID NO: 163 - DNA, 50 bp, Artificial Sequence
  Oligonucleotide
gcacccagcggaatgtgcttagtatttggtcaccagccgtcatcctgggc

SEQ ID NO: 164 - DNA, 50 bp, Artificial Sequence
  Oligonucleotide
tgcagctgtctaggtctgcggccacatcttggggacacactggactgttc

SEQ ID NO: 165 - DNA, 50 bp, Artificial Sequence
  Oligonucleotide
aagagcacggtcccccaggaggcagctcaggataggtggtatggagctgt

SEQ ID NO: 166 - DNA, 50 bp, Artificial Sequence
  Oligonucleotide
agcgtttggtgttaccttctcctgggaggtcctgctgcaactcaagttcc

SEQ ID NO: 167 - DNA, 50 bp, Artificial Sequence
  Oligonucleotide
agtgattttggtggccagtaaatgccagccatttctcaaacccacctcgg

SEQ ID NO: 168 - DNA, 50 bp, Artificial Sequence
  Oligonucleotide
gcctctctccctggacatacgttagcacattggcattcagtattggtggc

SEQ ID NO: 169 - DNA, 50 bp, Artificial Sequence
  Oligonucleotide
gaaggcggtgcaggtcctcatggtgctctccctcattctctgctgtctct

SEQ ID NO: 170 - DNA, 50 bp, Artificial Sequence
  Oligonucleotide
gaacttctacagaagccaagctccctggagccctgttggcagctctagct

SEQ ID NO: 171 - DNA, 50 bp, Artificial Sequence
  Oligonucleotide
gtgcaagcgccacggacctgggactcagcaccgataactcagacttgaat

SEQ ID NO: 172 - DNA, 50 bp, Artificial Sequence
  Oligonucleotide
tcttctgtgtccctaaggcctggtacagtgccaagcacatacttggtatc

SEQ ID NO: 173 - DNA, 50 bp, Artificial Sequence
  Oligonucleotide
gttcgacacccagtacccctacggtgagaagcaggatgagttcaagcgtc

SEQ ID NO: 174 - DNA, 50 bp, Artificial Sequence
  Oligonucleotide
gctggtctggggatagctggagcacttactcaggtggctggtgaaatgac

SEQ ID NO: 175 - DNA, 50 bp, Artificial Sequence
  Oligonucleotide
gaccttgcaatttagaatcaagcaggtgagacagggagaagtatgcctgc

SEQ ID NO: 176 - DNA, 50 bp, Artificial Sequence
  Oligonucleotide
cctggaaaaatggataaaggcgagcaccgtcaggagcgcagagatcggcc

SEQ ID NO: 177 - DNA, 50 bp, Artificial Sequence
  Oligonucleotide
cctggacctttgatggaacagatgggaggaagaagaggaggacgtggagg

SEQ ID NO: 178 - DNA, 50 bp, Artificial Sequence
  Oligonucleotide
gggaaccccttgtgagcatgctcagtatcattgtggagaaccaagagggc

SEQ ID NO: 179 - DNA, 50 bp, Artificial Sequence
  Oligonucleotide
gtgcgagggtagatggttcctgcacacagaagttaccacaggggtcaggt

SEQ ID NO: 180 - DNA, 50 bp, Artificial Sequence
  Oligonucleotide
actcaaagctaaggagcagtcaggaacccagataagaaagccatcctagt

SEQ ID NO: 181 - DNA, 50 bp, Artificial Sequence
  Oligonucleotide
gacagaggcttcaggtacaactggccacagagatagtcctggaagacacg

SEQ ID NO: 182 - DNA, 50 bp, Artificial Sequence
  Oligonucleotide
gacaccgtgctcccgtctctcaggcagcgaagttcgatccatcaaccaaa

SEQ ID NO: 183 - DNA, 50 bp, Artificial Sequence
  Oligonucleotide
ggggccctggtaggctcctttagaaggaccatttctgttcctagagctta

SEQ ID NO: 184 - DNA, 50 bp, Artificial Sequence
  Oligonucleotide
gtgcctggtgccaggtacacggtcctcttctcgcacggcaatgccgtgga

SEQ ID NO: 185 - DNA, 50 bp, Artificial Sequence
  Oligonucleotide
ccagaaacctcttgtgttcttgcctaggcccaggtgttcctggcagccaa

SEQ ID NO: 186 - DNA, 50 bp, Artificial Sequence
  Oligonucleotide
catccccagagacctcttgtgttcctgccacatagctgccagggcttaag

SEQ ID NO: 187 - DNA, 50 bp, Artificial Sequence
  Oligonucleotide
gctgcattgctctgctgagctgtattgaaaccatgactgggcccactgtc

SEQ ID NO: 188 - DNA, 50 bp, Artificial Sequence
  Oligonucleotide
ggctgactcccagccctgacttgaaaccattagcgctaacttgctctgtt

SEQ ID NO: 189 - DNA, 50 bp, Artificial Sequence
  Oligonucleotide
gaggccctgactaccctggaagtagcaggccgcatgcttggaggtaaagt

SEQ ID NO: 190 - DNA, 50 bp, Artificial Sequence
  Oligonucleotide
gactcctgccccggttcaaccctaccagcttgtggtaacttactgtcaca

SEQ ID NO: 191 - DNA, 50 bp, Artificial Sequence
  Oligonucleotide
gccatgtcaccgagccccattgattcccagagggtcttagtcctggaaag

SEQ ID NO: 192 - DNA, 50 bp, Artificial Sequence
  Oligonucleotide
tccccatcttcctggttctgctccttcgtggcctagtcttgtggacacca

SEQ ID NO: 193 - DNA, 50 bp, Artificial Sequence
  Oligonucleotide
cctgggcatggaatcctgtggcatccacaaaactaccttcaactccatag

SEQ ID NO: 194 - DNA, 50 bp, Artificial Sequence
  Oligonucleotide
gttctacaacttgcacagggtaacagaggaagtggctgaggcctagagtc

SEQ ID NO: 195 - DNA, 50 bp, Artificial Sequence
  Oligonucleotide
gctgtggtcagtggcttagctcggacaaggagatgagagcccatgtgttg

SEQ ID NO: 196 - DNA, 50 bp, Artificial Sequence
  Oligonucleotide
tggaggcattttgctgtgtgaggccgatcgccactgtaaaggtcctagag

SEQ ID NO: 197 - DNA, 50 bp, Artificial Sequence
  Oligonucleotide
gaatgctggtgagatgcttcatgggactgggggtctcctgctcagtctgg

SEQ ID NO: 198 - DNA, 50 bp, Artificial Sequence
  Oligonucleotide
cttttgggtgtggggcaggcagagagggatggtgtccagagatacatcac

SEQ ID NO: 199 - DNA, 50 bp, Artificial Sequence
  Oligonucleotide
cagcggaaccgcccaggatcagattgcatgtgactctgaagctgacgaac

SEQ ID NO: 200 - DNA, 50 bp, Artificial Sequence
  Oligonucleotide
gctggaaaaaggaccctgaagaacgccccacttttgagtacttgcagagc

SEQ ID NO: 201 - DNA, 50 bp, Artificial Sequence
  Oligonucleotide
gttgtgtctggagaagaagctgggtcaggggtgtttcgctgaagtgtggc

SEQ ID NO: 202 - DNA, 50 bp, Artificial Sequence
  Oligonucleotide
gccgcctagtagttccctgtcacaaagggatgccaaggcttaccgatctg

SEQ ID NO: 203 - DNA, 50 bp, Artificial Sequence
  Oligonucleotide
gttttgtacagtgcctggcactctgtgggtgctcaataaatggataggag

SEQ ID NO: 204 - DNA, 50 bp, Artificial Sequence
  Oligonucleotide
ctctgggtcaatcggaaggcgctatgccaggactgatgagattggcgtgg

SEQ ID NO: 205 - DNA, 50 bp, Artificial Sequence
  Oligonucleotide
gccatgctgagagctgggctttcctctgtgaccatcccggcctgtaacat

SEQ ID NO: 206 - DNA, 50 bp, Artificial Sequence
  Oligonucleotide
gctgagagctgggctttcctctgtgaccatcccggcctgtaacatatctg

SEQ ID NO: 207 - DNA, 50 bp, Artificial Sequence
  Oligonucleotide
gcctgtccagctccctctccccaagaaacaacatgaatgagcaacttcag

SEQ ID NO: 208 - DNA, 50 bp, Artificial Sequence
  Oligonucleotide
agttggtcttggtgtcatatggatcagaggcacaagtgcagaggctgtgg

SEQ ID NO: 209 - DNA, 50 bp, Artificial Sequence
  Oligonucleotide
tcctcaggtgactggggacttggaaccctaggacctgaacaaccaagact

SEQ ID NO: 210 - DNA, 50 bp, Artificial Sequence
  Oligonucleotide
gcgcatgcaccttcgtcagtacgagctgctctaagaagggaacccccaaa

SEQ ID NO: 211 - DNA, 50 bp, Artificial Sequence
  Oligonucleotide
gctgcctcctcctggctgtttttgtgcctgtttgaagctactgctgcctc

SEQ ID NO: 212 - DNA, 50 bp, Artificial Sequence
  Oligonucleotide
ggtgccacagttttaaaccagaaggtggcactctgtggctccttgtagta

SEQ ID NO: 213 - DNA, 50 bp, Artificial Sequence
  Oligonucleotide
gccccaggagctgctgagacggctgaaaagtcttccactaagaaggcagt

SEQ ID NO: 214 - DNA, 50 bp, Artificial Sequence
  Oligonucleotide
ctaaaggaagggcctctgctgactcctaccagagcatccgtccagctcag

SEQ ID NO: 215 - DNA, 50 bp, Artificial Sequence
  Oligonucleotide
ctggatcagagaccctgcctctgtttgaccccgcactgactgaataaagc

SEQ ID NO: 216 - DNA, 50 bp, Artificial Sequence
  Oligonucleotide
agcagcgcgaggccagagtccaataaactcgtgctcatctgcagcctcct

SEQ ID NO: 217 - DNA, 50 bp, Artificial Sequence
  Oligonucleotide
ggccctgccaccagaaagtcgagcactggtcctagtcaggctgtgatgaa

SEQ ID NO: 218 - DNA, 50 bp, Artificial Sequence
  Oligonucleotide
gccaacattcagtctggtatgtgaggcgtgcgtgaagcaagaactcctgg

SEQ ID NO: 219 - DNA, 50 bp, Artificial Sequence
  Oligonucleotide
gtagattgctggcctgttgtaggtggtagggacacagatgaccgacctgg

SEQ ID NO: 220 - DNA, 50 bp, Artificial Sequence
  Oligonucleotide
ggccacacagccaagaagagcccatgcaactcagtccaggattttactgg

SEQ ID NO: 221 - DNA, 50 bp, Artificial Sequence
  Oligonucleotide
ttcctggcctcccctgagtacgtgaacctccccatcaatggcaacgggaa

SEQ ID NO: 222 - DNA, 50 bp, Artificial Sequence
  Oligonucleotide
tctagggctagtacttagtttcacacccgggagctgggagaaaaaacctg

SEQ ID NO: 223 - DNA, 50 bp, Artificial Sequence
  Oligonucleotide
gagcactcaacccagaaggcgaagatagcttttggttgtaggcggcttcc

SEQ ID NO: 224 - DNA, 50 bp, Artificial Sequence
  Oligonucleotide
aggtggcagcagccatccgttattatttccaatggagacctagcccaggc

SEQ ID NO: 225 - DNA, 50 bp, Artificial Sequence
  Oligonucleotide
tctttatggtgggggcagactttgcacttactgcagtgcaacacttgcac

SEQ ID NO: 226 - DNA, 50 bp, Artificial Sequence
  Oligonucleotide
aattagccgggcgtggtggcaggctcctcgggaggctgaggcagaaaaat

SEQ ID NO: 227 - DNA, 50 bp, Artificial Sequence
  Oligonucleotide
ccaggaactgagcctagggagctcatctggcagcaatggcttttactcat

SEQ ID NO: 228 - DNA, 50 bp, Artificial Sequence
  Oligonucleotide
cagcgaagtccgctcccgcgcccaagaagggctcgaagaaagccgtgact

SEQ ID NO: 229 - DNA, 50 bp, Artificial Sequence
  Oligonucleotide
cccactggggggttggggtaatattctgtggtcctcagccctgtacctta

SEQ ID NO: 230 - DNA, 50 bp, Artificial Sequence
  Oligonucleotide
cgtgtgtccgtggaaccagtcctagccgcgtgtgacagtcttgcattctg

SEQ ID NO: 231 - DNA, 50 bp, Artificial Sequence
  Oligonucleotide
ctgatgtgtctctcacagcttgaaaagcctgagacagctgtcttgtgagg

SEQ ID NO: 232 - DNA, 50 bp, Artificial Sequence
  Oligonucleotide
accgtcctcatcataaagtctctgcgttctggccatgacccccgggccca

SEQ ID NO: 233 - DNA, 50 bp, Artificial Sequence
  Oligonucleotide
cttggatggacccacgaacgctcttagctttctcagggggtcagcagagt

SEQ ID NO: 234 - DNA, 50 bp, Artificial Sequence
  Oligonucleotide
tacgactactcgccctatggctattacggctacggccccggctacgacta

SEQ ID NO: 235 - DNA, 50 bp, Artificial Sequence
  Oligonucleotide
gcggcagcaggagcgaccaactgatcgcacacatgctttgtttggatatg

SEQ ID NO: 236 - DNA, 50 bp, Artificial Sequence
  Oligonucleotide
ccagagctctaggtgtttaggcagcgtgtggtgtctgagaggccatagcg

SEQ ID NO: 237 - DNA, 50 bp, Artificial Sequence
  Oligonucleotide
ggtgaccagcagagtggttatgggaaggtatccaggcgaggtggtcatca

SEQ ID NO: 238 - DNA, 50 bp, Artificial Sequence
  Oligonucleotide
cccccagtattgtagagcaagtcttgtgttaaaagcccagtgtgacagtg

SEQ ID NO: 239 - DNA, 50 bp, Artificial Sequence
  Oligonucleotide
gcctgccggatgatgaatggcatgaagctgagtggccgagagattgacgt

SEQ ID NO: 240 - DNA, 50 bp, Artificial Sequence
  Oligonucleotide
acaaccaacagaactggggttcccaacccatcgctcagcagccgcttcag

SEQ ID NO: 241 - DNA, 50 bp, Artificial Sequence
  Oligonucleotide
caagataagcatttctttcctgagttcaggtgactgaggaagagccacaa

SEQ ID NO: 242 - DNA, 50 bp, Artificial Sequence
  Oligonucleotide
tcccactgcctcctctccagtggtctcccaggtgccagacccaaaagctt

SEQ ID NO: 243 - DNA, 50 bp, Artificial Sequence
  Oligonucleotide
tccagaaactggggatggaatctagacttgtgagcggcggtggtgcctgc

SEQ ID NO: 244 - DNA, 50 bp, Artificial Sequence
  Oligonucleotide
cctggaggcttgcttgggactggaggcttgcttggacagttcctctgtgt

SEQ ID NO: 245 - DNA, 50 bp, Artificial Sequence
  Oligonucleotide
ctgtgcagcagtggctctgtgtgtaaatgctatgcactgaggatacacaa

SEQ ID NO: 246 - DNA, 50 bp, Artificial Sequence
  Oligonucleotide
aagcctttgtatgtgtcctcagggggcagaccgactttaagagggaccag

SEQ ID NO: 247 - DNA, 50 bp, Artificial Sequence
  Oligonucleotide
ctgaggcatcctgctgtcatgggaaggtctccgcccaaatgtcagatgca

SEQ ID NO: 248 - DNA, 50 bp, Artificial Sequence
  Oligonucleotide
gaccatccgaaacctgcgtccctggtgatgttctcaagcctcggaagtgg

SEQ ID NO: 249 - DNA, 50 bp, Artificial Sequence
  Oligonucleotide
ggagattcacagcaactgatcaaagggagtccagtcaacgtgagcaagcg

SEQ ID NO: 250 - DNA, 50 bp, Artificial Sequence
  Oligonucleotide
aatgctgcagttcctgatgagatcccccctctcgagggcgatgaggatgc

SEQ ID NO: 251 - DNA, 50 bp, Artificial Sequence
  Oligonucleotide
gtcccaagtatttccagcaggaagtaatgtcttcctcagcctcaaccagg

SEQ ID NO: 252 - DNA, 50 bp, Artificial Sequence
  Oligonucleotide
gaacaccacggacttcctcgacaccatcaagagcaacctggacagagccc

SEQ ID NO: 253 - DNA, 50 bp, Artificial Sequence
  Oligonucleotide
gggatttagccaagagcacagacttggattccttctgtccctccccacct

SEQ ID NO: 254 - DNA, 50 bp, Artificial Sequence
  Oligonucleotide
tggaagatcagacccagctccttacccttgtctgccagttgtaccagggc

SEQ ID NO: 255 - DNA, 50 bp, Artificial Sequence
  Oligonucleotide
gaaccgacagaacatgggctgatcttcccacaacaccacaggactgcagg

SEQ ID NO: 256 - DNA, 50 bp, Artificial Sequence
  Oligonucleotide
ggggcccttaggccgttgggactttgatacccaggaagaatacagcgagt

SEQ ID NO: 257 - DNA, 50 bp, Artificial Sequence
  Oligonucleotide
gcagctgcttcggatccacactgtatctgtgtcatccccacatgggtcct

SEQ ID NO: 258 - DNA, 50 bp, Artificial Sequence
  Oligonucleotide
ggggctccacacctttgctgtgtgttctggggcaacctactaatcctctc

SEQ ID NO: 259 - DNA, 50 bp, Artificial Sequence
  Oligonucleotide
caagaggggcgggctcagagctttgtcacttgccacatggtgtctcccaa

SEQ ID NO: 260 - DNA, 50 bp, Artificial Sequence
  Oligonucleotide
ggattcatcctgggagagggggcaaggtggaatgcagataactcacatgt

SEQ ID NO: 261 - DNA, 50 bp, Artificial Sequence
  Oligonucleotide
cccaaaggccacatccaagacaggcaataatgagcagagtttacagctcc

SEQ ID NO: 262 - DNA, 50 bp, Artificial Sequence
  Oligonucleotide
cctcaacaggcccagggagggaagtgtgagcgccttggtatgacttaaaa

SEQ ID NO: 263 - DNA, 50 bp, Artificial Sequence
  Oligonucleotide
ggagggcttgaggttggtgaggttaggtgcgtgtttcctgtgcaagtcag

SEQ ID NO: 264 - DNA, 50 bp, Artificial Sequence
  Oligonucleotide
ggagacttgaggagggcttgaggttggtgaggttaggtgcgtgtttcctg

SEQ ID NO: 265 - DNA, 50 bp, Artificial Sequence
  Oligonucleotide
attgcctctgacgtctggtcttttggagtcactctgcatgagctgctgac

SEQ ID NO: 266 - DNA, 50 bp, Artificial Sequence
  Oligonucleotide
aaaacttctgtactgccctggaatatgggctgccccccacagctggctgg

SEQ ID NO: 267 - DNA, 50 bp, Artificial Sequence
  Oligonucleotide
cctctgtgcctgagttctccctgttgtctcaaagcggtacccatcctccc

SEQ ID NO: 268 - DNA, 50 bp, Artificial Sequence
  Oligonucleotide
gccctgccggaagcagattgaccagcagaactgtacctgttgaggcactt

SEQ ID NO: 269 - DNA, 50 bp, Artificial Sequence
  Oligonucleotide
cctggacagtgtggagtgttacgacccagatacagacacctggagcgagg

SEQ ID NO: 270 - DNA, 50 bp, Artificial Sequence
  Oligonucleotide
gcagatgttgtgggtgcccttctgttcctggaggattatgttcggtacac

SEQ ID NO: 271 - DNA, 50 bp, Artificial Sequence
  Oligonucleotide
ctgggccctgtaaaatagtgttactgtaatactctgttttgcctcctgcc

SEQ ID NO: 272 - DNA, 50 bp, Artificial Sequence
  Oligonucleotide
tcctgcgtctgtcttccctgcttttcagtcgtcgggcttagagaagcctg

SEQ ID NO: 273 - DNA, 50 bp, Artificial Sequence
  Oligonucleotide
gtcaccctgtgcttcctgcccaagatactgacccattgaacccccaaagc

SEQ ID NO: 274 - DNA, 50 bp, Artificial Sequence
  Oligonucleotide
ttgcttgtgtgcatgtgttgggtgcatgcttccgggtctcagctgcccca

SEQ ID NO: 275 - DNA, 50 bp, Artificial Sequence
  Oligonucleotide
cggcacgtccttggcgtctctaatgtctgcagctcaagggctggcacttt

SEQ ID NO: 276 - DNA, 50 bp, Artificial Sequence
  Oligonucleotide
ccaacaaagatgaagttccctatctacggaaaggcatgactgggaggccc

SEQ ID NO: 277 - DNA, 50 bp, Artificial Sequence
  Oligonucleotide
tctggcaccctcctgaatggaaccccagagtacctcctgtgtggaagggt

SEQ ID NO: 278 - DNA, 50 bp, Artificial Sequence
  Oligonucleotide
gtgtgtgcgggcatggatgtgactgggagctctgctgggcacccacatct

SEQ ID NO: 279 - DNA, 50 bp, Artificial Sequence
  Oligonucleotide
cctcttccaagccctgcgtccagcgagcgtcacagcacaacctgcaaaaa

SEQ ID NO: 280 - DNA, 50 bp, Artificial Sequence
  Oligonucleotide
caagctgaccatttcgaagcttgctcctggtgggcacgtgggacgtttct

SEQ ID NO: 281 - DNA, 50 bp, Artificial Sequence
  Oligonucleotide
accctctgcgaccggaggactatgcccctactcccggaagccctcggaca

SEQ ID NO: 282 - DNA, 50 bp, Artificial Sequence
  Oligonucleotide
gtggtagatcacttgaggtcaagagttgtgacaccagcctggccaacctg

SEQ ID NO: 283 - DNA, 50 bp, Artificial Sequence
  Oligonucleotide
tgatggttcttgctgggcagcttggagaaggcgtgatactctccagctcc

SEQ ID NO: 284 - DNA, 50 bp, Artificial Sequence
  Oligonucleotide
tagaactattattgaccacgcctcctccaagtcccagcgagcccgtgtac

SEQ ID NO: 285 - DNA, 50 bp, Artificial Sequence
  Oligonucleotide
gaatgagctcagtgaccacttggatgctatggactccaacctggataacc

SEQ ID NO: 286 - DNA, 50 bp, Artificial Sequence
  Oligonucleotide
cacttaacatagtgacctccagttccatccatgctgtcgcaagtgacagg

SEQ ID NO: 287 - DNA, 50 bp, Artificial Sequence
  Oligonucleotide
agataaaggggccaccaaggagtcgagtgagaaggatcgcggccgggaca

SEQ ID NO: 288 - DNA, 50 bp, Artificial Sequence
  Oligonucleotide
tgcagccagtcactgcacctccgtcctaccctcctaccagctatgatcag

SEQ ID NO: 289 - DNA, 50 bp, Artificial Sequence
  Oligonucleotide
cagagctattgcaccatgagcgtccttcctccttcctctccgggctgcca

SEQ ID NO: 290 - DNA, 50 bp, Artificial Sequence
  Oligonucleotide
ccctttcctctccctcagaatttgtgtttgctgcctctatcttgtttttt

SEQ ID NO: 291 - DNA, 50 bp, Artificial Sequence
  Oligonucleotide
tcctttccaactctacctccctcactcgagctcctttcccctgatcagag

SEQ ID NO: 292 - DNA, 50 bp, Artificial Sequence
  Oligonucleotide
ccacgcgcgaaaattcggccagggttctcgctcttgtcgcgtctgctcaa

SEQ ID NO: 293 - DNA, 50 bp, Artificial Sequence
  Oligonucleotide
agtggcgaggatggcaagaaaagctggcaacttctatgtacctgcagaac

SEQ ID NO: 294 - DNA, 50 bp, Artificial Sequence
  Oligonucleotide
gtagaaggtggagatgctggcaacagggaggaccaggtcaacaggcttat

SEQ ID NO: 295 - DNA, 50 bp, Artificial Sequence
  Oligonucleotide
agccttgcatgtgcagaaagtaaaagccagggtaggcttgtaacctgccc

SEQ ID NO: 296 - DNA, 50 bp, Artificial Sequence
  Oligonucleotide
tttgctcgaagccttcagtccgttgcagaggagcgagttggacgccactg

SEQ ID NO: 297 - DNA, 50 bp, Artificial Sequence
  Oligonucleotide
agggtcctgaattcttactgggttggtgaagattccacatacaaattttt

SEQ ID NO: 298 - DNA, 50 bp, Artificial Sequence
  Oligonucleotide
tgcttccaactgcgggacagggagtggccgtagcggcttgttggataagt

SEQ ID NO: 299 - DNA, 50 bp, Artificial Sequence
  Oligonucleotide
cggagacggcaaatggcggacttcgacacctacgacgatcgggcctacag

SEQ ID NO: 300 - DNA, 50 bp, Artificial Sequence
  Oligonucleotide
tggcttcacggctggctatgtggacagcaagagtcgttttcgcggaagcc

SEQ ID NO: 301 - DNA, 50 bp, Artificial Sequence
  Oligonucleotide
ccctcctgtgagagtctgaaggatactattgccagagctctgccttctgg

SEQ ID NO: 302 - DNA, 50 bp, Artificial Sequence
  Oligonucleotide
gtgcgacctcgatctgtcccaagcattcatgttccctcaccagctgtagt

SEQ ID NO: 303 - DNA, 50 bp, Artificial Sequence
  Oligonucleotide
ggacccggacaccctgtgggacctggcctcaaactcaccaaatcgctcat

SEQ ID NO: 304 - DNA, 50 bp, Artificial Sequence
  Oligonucleotide
gtcatcaagagccgctgccactggtcctccgtttactgacctggctgtgt

SEQ ID NO: 305 - DNA, 50 bp, Artificial Sequence
  Oligonucleotide
tgagcagggacagtcattttttaaatgtttttggccgggcgtggtggctc

SEQ ID NO: 306 - DNA, 50 bp, Artificial Sequence
  Oligonucleotide
tgggagcccagaagaaatgctcttttgcttggagtttgtcatcctacacc

SEQ ID NO: 307 - DNA, 50 bp, Artificial Sequence
  Oligonucleotide
cagccagtgccaacttcgctgccaactttggtgccattggtttcttctgg

SEQ ID NO: 308 - DNA, 50 bp, Artificial Sequence
  Oligonucleotide
gtgtctgggccctggagctgggatgacattgagtttgagctgctgacctg

SEQ ID NO: 309 - DNA, 50 bp, Artificial Sequence
  Oligonucleotide
tgctgtgtgactatgattcctaagatttccagggcttaagggctaacttc

SEQ ID NO: 310 - DNA, 50 bp, Artificial Sequence
  Oligonucleotide
ggccctggttgaaaagtactcatctcctggtctgacatccaaagagtcac

SEQ ID NO: 311 - DNA, 50 bp, Artificial Sequence
  Oligonucleotide
gcaggattctgcaaaatgtgtctcacccactactgagattgttcagcccc

SEQ ID NO: 312 - DNA, 50 bp, Artificial Sequence
  Oligonucleotide
cctgtttggatcctggtcctttttaactgttccttggtaattctgagcat

SEQ ID NO: 313 - DNA, 50 bp, Artificial Sequence
  Oligonucleotide
cctgagccagaagtggggtgcttatactcccaaaccttgagtgtccagcc

SEQ ID NO: 314 - DNA, 50 bp, Artificial Sequence
  Oligonucleotide
caggatgacaatcaggtcatggtgtctgagggcatcatcttcctcatctg

SEQ ID NO: 315 - DNA, 50 bp, Artificial Sequence
  Oligonucleotide
ggctttgggtggttccaattggtggagagaagctctgaggcacgtcatgc

SEQ ID NO: 316 - DNA, 50 bp, Artificial Sequence
  Oligonucleotide
ccatctgctggcacctgaggagagtgagcagcctggaccacaagcccagt

SEQ ID NO: 317 - DNA, 50 bp, Artificial Sequence
  Oligonucleotide
cctctagcggcttccagttccccgctcctgactcctgacctccaggatgt

SEQ ID NO: 318 - DNA, 50 bp, Artificial Sequence
  Oligonucleotide
tgcagaaggaatggtggatccaagtctcaatcccatttcagcctttcgac

SEQ ID NO: 319 - DNA, 50 bp, Artificial Sequence
  Oligonucleotide
cttctgccatgattgtgaggcctccccagccatgtggaactgtgaatcca

SEQ ID NO: 320 - DNA, 50 bp, Artificial Sequence
  Oligonucleotide
gtaggggtttccagcttccccaggctccggccttgtcagtctctttgcat

SEQ ID NO: 321 - DNA, 50 bp, Artificial Sequence
  Oligonucleotide
ccctctgtggttctgactggagaccccagtgtgggggaggtcttaccatt

SEQ ID NO: 322 - DNA, 50 bp, Artificial Sequence
  Oligonucleotide
caagatggcagcggcgctgcgcgtgcgttgttgagtgttcgggacgccgg

SEQ ID NO: 323 - DNA, 50 bp, Artificial Sequence
  Oligonucleotide
ggcctgcaggcgccatggtcttcctcaccgcgcagctctggctgcggaat

SEQ ID NO: 324 - DNA, 50 bp, Artificial Sequence
  Oligonucleotide
tgctgctcctgctgccccatgagctgtgccaagtgtgcccagggctgcat

SEQ ID NO: 325 - DNA, 50 bp, Artificial Sequence
  Oligonucleotide
gaacccgcgtgcaacctgtcccgactctagccgcctcttcagcacgccat

SEQ ID NO: 326 - DNA, 50 bp, Artificial Sequence
  Oligonucleotide
agaaggagggtttctggctgtggttctaaatggagccccaggaagctgcc

SEQ ID NO: 327 - DNA, 50 bp, Artificial Sequence
  Oligonucleotide
ctatccagaagaagctggctgcaaaagggctaagggatccatggggccgc

SEQ ID NO: 328 - DNA, 50 bp, Artificial Sequence
  Oligonucleotide
ccagaagaagctggctgcaaaagggctaagggatccatggggccgcaatg

SEQ ID NO: 329 - DNA, 50 bp, Artificial Sequence
  Oligonucleotide
ctgagcctgggtgctcactgtggcggtccccgtcctggctatgaaacctt

SEQ ID NO: 330 - DNA, 50 bp, Artificial Sequence
  Oligonucleotide
cccatataagctgctgccactgcagaggtttttacctcgacctccaggtg

SEQ ID NO: 331 - DNA, 50 bp, Artificial Sequence
  Oligonucleotide
agctccgggtggctggttctcagtggttgtctcatgtctctttttctgtc

SEQ ID NO: 332 - DNA, 50 bp, Artificial Sequence
  Oligonucleotide
cccggttcattttatgcgtgcgagaagtcagtggtaactgctgcagggct

SEQ ID NO: 333 - DNA, 50 bp, Artificial Sequence
  Oligonucleotide
tatcacaattgccacccatcgggttttgggtgtgtgttttcatagcgtgg

SEQ ID NO: 334 - DNA, 50 bp, Artificial Sequence
  Oligonucleotide
gccagtcttttttcattgacgccccagattccccagccacgttagcctac

SEQ ID NO: 335 - DNA, 50 bp, Artificial Sequence
  Oligonucleotide
catcaggagaaaggctgggtcttgggaccttgtcctccccagttggccta

SEQ ID NO: 336 - DNA, 50 bp, Artificial Sequence
  Oligonucleotide
cagcttccagtggtggccgtagacttggctcggaacttagtggcaccaga

SEQ ID NO: 337 - DNA, 50 bp, Artificial Sequence
  Oligonucleotide
cgaggaccgcgactctcaactccgaagtcaagcccgcactctgattacct

SEQ ID NO: 338 - DNA, 50 bp, Artificial Sequence
  Oligonucleotide
agagagacttcctgaagcagcgccccaccaagctcaagagcctcatccgc

SEQ ID NO: 339 - DNA, 50 bp, Artificial Sequence
  Oligonucleotide
ctcctgcttcctccctgccattcatccctgcccctctccatgaagcttga

SEQ ID NO: 340 - DNA, 50 bp, Artificial Sequence
  Oligonucleotide
aaagggttggaggcagctggcacaagaggctgaggcctggctgaattacc

SEQ ID NO: 341 - DNA, 50 bp, Artificial Sequence
  Oligonucleotide
ggctattcctccccagaacctgacattcaagactcctctggaagtgaagc

SEQ ID NO: 342 - DNA, 50 bp, Artificial Sequence
  Oligonucleotide
gttgccaactgttgttccagccatccacacaggagtctgttctgaggtgg

SEQ ID NO: 343 - DNA, 50 bp, Artificial Sequence
  Oligonucleotide
cctggagatcagactgttgctttcgcatgatgtatgtagtgtctcatgac

SEQ ID NO: 344 - DNA, 50 bp, Artificial Sequence
  Oligonucleotide
ggggcccccaccttcaatgtcactgtcaccaagactgacaagacgctagt

SEQ ID NO: 345 - DNA, 50 bp, Artificial Sequence
  Oligonucleotide
ggctggatggacagacacctccccctacccatatccctcccgtgtgtggt

SEQ ID NO: 346 - DNA, 50 bp, Artificial Sequence
  Oligonucleotide
gctaatcccagtcggtgccgcatccccagcccgccgccatggccgcctac

SEQ ID NO: 347 - DNA, 50 bp, Artificial Sequence
  Oligonucleotide
attgtcaagcatctggagggtctctctgaagaggctatcatggagctgaa

SEQ ID NO: 348 - DNA, 50 bp, Artificial Sequence
  Oligonucleotide
ccatgttgaggggctccattcccaattcctgggtcaaggtgaattaaccc

SEQ ID NO: 349 - DNA, 50 bp, Artificial Sequence
  Oligonucleotide
cttgcagcagctctggtggcagctgtccttgaggaacctttggtgtgtgg

SEQ ID NO: 350 - DNA, 50 bp, Artificial Sequence
  Oligonucleotide
tagttggctttgtctgtcaggtgcagtctggcgggagtccaggaggcagc

SEQ ID NO: 351 - DNA, 50 bp, Artificial Sequence
  Oligonucleotide
tcctcggacccgagaacccgaaaattgccaaggacccatcctggatcatc

SEQ ID NO: 352 - DNA, 50 bp, Artificial Sequence
  Oligonucleotide
agcccaggtctaaatgtaatggttggtttattgttctataaccccagccc

SEQ ID NO: 353 - DNA, 50 bp, Artificial Sequence
  Oligonucleotide
ccacttgcacctctccacctttggcactagaactcctgagacaccacttc

SEQ ID NO: 354 - DNA, 50 bp, Artificial Sequence
  Oligonucleotide
gagtaacggctctgctgccagggtttctctgggctcattcttccactgac

SEQ ID NO: 355 - DNA, 50 bp, Artificial Sequence
  Oligonucleotide
tggctcggatcctcagtgcctgtgtcttgtcaaagcggtgcccgccagac

SEQ ID NO: 356 - DNA, 50 bp, Artificial Sequence
  Oligonucleotide
cgttcaaactgtccactctgatccaaccctgtactgatagtacttcccag

SEQ ID NO: 357 - DNA, 50 bp, Artificial Sequence
  Oligonucleotide
gtaatcctagcacttttgtcgcctgggcgacacaccaaggctctgtctca

SEQ ID NO: 358 - DNA, 50 bp, Artificial Sequence
  Oligonucleotide
ctccatggccctcggccgcttgcacccgctctctgttgtacactttcaat

SEQ ID NO: 359 - DNA, 50 bp, Artificial Sequence
  Oligonucleotide
tggccaagttcctccacaagcacgacttggacctcatctgccgagcacac

SEQ ID NO: 360 - DNA, 50 bp, Artificial Sequence
  Oligonucleotide
gccaagaaatagcccccgcacaccaccctgtgccccagatgatggattga

SEQ ID NO: 361 - DNA, 50 bp, Artificial Sequence
  Oligonucleotide
gctgcccctgagaagagacttaatccaagcctgattgtactagtggcatc

SEQ ID NO: 362 - DNA, 50 bp, Artificial Sequence
  Oligonucleotide
cgagttcctgaaaaaggagactgcacagcgtcgggttctggaggagtcgg

SEQ ID NO: 363 - DNA, 50 bp, Artificial Sequence
  Oligonucleotide
tccagtcctcacaacctgtccttcacctagtccctcctgacccagggatg

SEQ ID NO: 364 - DNA, 50 bp, Artificial Sequence
  Oligonucleotide
cttcttcctaggggcctcgtgatctgaggggtggtgcctacttccactgt

SEQ ID NO: 365 - DNA, 50 bp, Artificial Sequence
  Oligonucleotide
tgatcagctctgaggtgcaacttcttcacatactgtacatacctgtgacc

SEQ ID NO: 366 - DNA, 50 bp, Artificial Sequence
  Oligonucleotide
cagccctgtgtgtgaatcgtttgtgacgtgtgcaaatgggaaaggagggg

SEQ ID NO: 367 - DNA, 50 bp, Artificial Sequence
  Oligonucleotide
cccagaagaacctcaggaggtaaccttgggcccttccctgctatcctttt

SEQ ID NO: 368 - DNA, 50 bp, Artificial Sequence
  Oligonucleotide
gattcaccctgtccaaactgcctaagccctccgccattctcaagccctgc

SEQ ID NO: 369 - DNA, 50 bp, Artificial Sequence
  Oligonucleotide
gggaacggatgtggaaggaagaactgtcaccctcttaaggcccagggtcg

SEQ ID NO: 370 - DNA, 50 bp, Artificial Sequence
  Oligonucleotide
ggagaagctgaatgcaacaaacattgagctagccacagtgcagcctggcc

SEQ ID NO: 371 - DNA, 50 bp, Artificial Sequence
  Oligonucleotide
accctggagctagtggaggaaactgtgcaggctatggaggtggagtaagc

SEQ ID NO: 372 - DNA, 50 bp, Artificial Sequence
  Oligonucleotide
tgcagcccacggctatggtgccttcctgactctcagtatcctcgaccgat

SEQ ID NO: 373 - DNA, 50 bp, Artificial Sequence
  Oligonucleotide
accagtgaagacattgaggagctggtggaacctgtggcagcacatggccc

SEQ ID NO: 374 - DNA, 50 bp, Artificial Sequence
  Oligonucleotide
accgggacatccggctgatggtcatggagatccgcaatgcttatgctgtg

SEQ ID NO: 375 - DNA, 50 bp, Artificial Sequence
  Oligonucleotide
gctcagcttctccacaaggctagaaatggggcacagagccactggaggcc

SEQ ID NO: 376 - DNA, 50 bp, Artificial Sequence
  Oligonucleotide
tgattttggggtagcaatccaggagaaggtgctggagagggtgaatgccg

SEQ ID NO: 377 - DNA, 50 bp, Artificial Sequence
  Oligonucleotide
ctgcagcttcgtagtactcccttccggtacctacttacaccttccatgca

SEQ ID NO: 378 - DNA, 50 bp, Artificial Sequence
  Oligonucleotide
taatctggacattcgaggaattggccgctgtcactgcttgttgtttgcgc

SEQ ID NO: 379 - DNA, 50 bp, Artificial Sequence
  Oligonucleotide
accctgcagagctatggtgaggtgtggataaggcttaggtgccaggctgt

SEQ ID NO: 380 - DNA, 50 bp, Artificial Sequence
  Oligonucleotide
ggagaagagcaagggttccctcaagaggaagtgagcggtgctgtcctcag

SEQ ID NO: 381 - DNA, 50 bp, Artificial Sequence
  Oligonucleotide
cctagtcttccttcatccttgccctctgttggcacaggcattatctctgc

SEQ ID NO: 382 - DNA, 50 bp, Artificial Sequence
  Oligonucleotide
cctgtcccatgttggaagttgctctgaaggggtggtagatgctggaagcc

SEQ ID NO: 383 - DNA, 50 bp, Artificial Sequence
  Oligonucleotide
ggtgtcatgttggatcgctttgtgactgttcatctgtccttgacagtggc

SEQ ID NO: 384 - DNA, 50 bp, Artificial Sequence
  Oligonucleotide
aatctccccagccgacttccactgggctgacagactttgctgaccacagg

SEQ ID NO: 385 - DNA, 50 bp, Artificial Sequence
  Oligonucleotide
ccctgtagtccagtggtgctgccctgttgtgcaaactgctcctttttctc

SEQ ID NO: 386 - DNA, 50 bp, Artificial Sequence
  Oligonucleotide
gctttgagacctttcctctcctgggtactgaggtgctatgaagccaactg

SEQ ID NO: 387 - DNA, 50 bp, Artificial Sequence
  Oligonucleotide
agagtcgcggggacacaggagtcttcctacagtacacacacgcccgcctc

SEQ ID NO: 388 - DNA, 50 bp, Artificial Sequence
  Oligonucleotide
catcctaaaaatggggtccaggcagacccctccagacctcacactgccga

SEQ ID NO: 389 - DNA, 50 bp, Artificial Sequence
  Oligonucleotide
gctcctgctgcaaccgctgtgaatgctgctgagaacctccctctatgggg

SEQ ID NO: 390 - DNA, 50 bp, Artificial Sequence
  Oligonucleotide
ggctcacacagcttaagagtagctgtctctcaaacgtgcgctcacagttg

SEQ ID NO: 391 - DNA, 50 bp, Artificial Sequence
  Oligonucleotide
agcttcgcaagagcatgtggaaggaccggaatctggacgtggtccgcaag

SEQ ID NO: 392 - DNA, 50 bp, Artificial Sequence
  Oligonucleotide
gtgcaaacagacattccagagagcctgatccacatccagcagcagagccc

SEQ ID NO: 393 - DNA, 50 bp, Artificial Sequence
  Oligonucleotide
gttgggaatgtgcttggcgctgaccctgcgggcatctgactggtcttcca

SEQ ID NO: 394 - DNA, 50 bp, Artificial Sequence
  Oligonucleotide
atacctgtggccccccacacaactgagcccatgctgatggagtaccctga

SEQ ID NO: 395 - DNA, 50 bp, Artificial Sequence
  Oligonucleotide
ctggcgaaccttggagagggaatgctgattgtcttgaccaaacccacagc

SEQ ID NO: 396 - DNA, 50 bp, Artificial Sequence
  Oligonucleotide
ccctcctttatgacctttgggacattgggaatacccagccaactctccac

SEQ ID NO: 397 - DNA, 50 bp, Artificial Sequence
  Oligonucleotide
ccatggacattgctgctcttggtggtgttatctaatttttgtgataggga

SEQ ID NO: 398 - DNA, 50 bp, Artificial Sequence
  Oligonucleotide
cagaaattcagaaagggagccagccaccctggggcagtgaagtgccactg

SEQ ID NO: 399 - DNA, 50 bp, Artificial Sequence
  Oligonucleotide
cccagggagtgctcgaggcgcatcaggcccgttttttaccagtttatatc

SEQ ID NO: 400 - DNA, 50 bp, Artificial Sequence
  Oligonucleotide
ctcagttcctaatatcccgctccttgctgagaccatctcctggggcaggg

SEQ ID NO: 401 - DNA, 50 bp, Artificial Sequence
  Oligonucleotide
tgctcgaggcgcatcaggcccgttttttaccagtttatatcacggtcttc

SEQ ID NO: 402 - DNA, 50 bp, Artificial Sequence
  Oligonucleotide
ataccggcttccagagaccccttttctccagccatattacatcaggctag

SEQ ID NO: 403 - DNA, 50 bp, Artificial Sequence
  Oligonucleotide
aattctcagggctctacccccctttcctggtcctaggtggccagtgggta

SEQ ID NO: 404 - DNA, 50 bp, Artificial Sequence
  Oligonucleotide
ccgcagctctcatcattgtgatgtgtagcatgtctgccctctgactggac

SEQ ID NO: 405 - DNA, 50 bp, Artificial Sequence
  Oligonucleotide
cactgtcgtccttcctcagagggcctcacgccaaacaaacggccttttcg

SEQ ID NO: 406 - DNA, 50 bp, Artificial Sequence
  Oligonucleotide
agcctggctctgtgctgcgggtgctctggttggccgactgcgatgtgagt

SEQ ID NO: 407 - DNA, 50 bp, Artificial Sequence
  Oligonucleotide
ggtcctgtacgacatttactggtctgaggagatggaggaccggctgcagg

SEQ ID NO: 408 - DNA, 50 bp, Artificial Sequence
  Oligonucleotide
gcaccgatgcacacaccgcaccccaccactgtactctgaaattggcgagt

SEQ ID NO: 409 - DNA, 50 bp, Artificial Sequence
  Oligonucleotide
gcattggggccaaacacagaatcagcaaagaggaggccatgcgctggttc

SEQ ID NO: 410 - DNA, 50 bp, Artificial Sequence
  Oligonucleotide
ttggggccaaacacagaatcagcaaagaggaggccatgcgctggttccag

SEQ ID NO: 411 - DNA, 50 bp, Artificial Sequence
  Oligonucleotide
caggggatttggggctttcttgaaagacagtccaagccctggataatgct

SEQ ID NO: 412 - DNA, 50 bp, Artificial Sequence
  Oligonucleotide
ggttacccactctgtccactcccataggctacagaaaaagtcacaagcgc

SEQ ID NO: 413 - DNA, 50 bp, Artificial Sequence
  Oligonucleotide
tcctttcggccggaaccgccatcttccagtaattcgccaaaatgacgaac

SEQ ID NO: 414 - DNA, 50 bp, Artificial Sequence
  Oligonucleotide
tccgggtggataaggcagctgctgcagcagcggcactacaagccaaatca

SEQ ID NO: 415 - DNA, 50 bp, Artificial Sequence
  Oligonucleotide
aggcagaggtccaagtaaaccgctagcttgttgcaccgtggaggccacag

SEQ ID NO: 416 - DNA, 50 bp, Artificial Sequence
  Oligonucleotide
ggagcgggctgctgagagctaaacccagcaattttctatgattttttcag

SEQ ID NO: 417 - DNA, 50 bp, Artificial Sequence
  Oligonucleotide
gccaacctccctgtccagatgcagctattttggtatctcctatcacatgc

SEQ ID NO: 418 - DNA, 50 bp, Artificial Sequence
  Oligonucleotide
ctggacaacaagctccgtgaagacctggagcgactgaagaagattcgggc

SEQ ID NO: 419 - DNA, 50 bp, Artificial Sequence
  Oligonucleotide
cctcgttgcactgctgagagcaagatgggtcaccagcagctgtactggag

SEQ ID NO: 420 - DNA, 50 bp, Artificial Sequence
  Oligonucleotide
agagcagcccaggtggtcatcagagtcaccaatgccaatgccagcctgca

SEQ ID NO: 421 - DNA, 50 bp, Artificial Sequence
  Oligonucleotide
gcgaaaattcggccagggttctcgctcttgtcgtgtctgttcaaaccggc

SEQ ID NO: 422 - DNA, 50 bp, Artificial Sequence
  Oligonucleotide
tggactaaatgctcttccttcagaggattatccggggcatctactcaatg

SEQ ID NO: 423 - DNA, 50 bp, Artificial Sequence
  Oligonucleotide
ctggggacgagacaggtgctaaagttgaacgagctgatggatatgaacca

SEQ ID NO: 424 - DNA, 50 bp, Artificial Sequence
  Oligonucleotide
ccaacaggtccgccaaatccggaagaagatgatggaaatcatgacccgag

SEQ ID NO: 425 - DNA, 50 bp, Artificial Sequence
  Oligonucleotide
gggacgagacaggtgctaaagttgaacgagctgatggatatgaaccacca

SEQ ID NO: 426 - DNA, 50 bp, Artificial Sequence
  Oligonucleotide
ggggcatgttgtgtcatgtagtcagccacttatgcaccaatgtgaggaaa

SEQ ID NO: 427 - DNA, 50 bp, Artificial Sequence
  Oligonucleotide
gtcctataaatgcacctcctgtcaaaaccatgcctgagaggtcccggctg

SEQ ID NO: 428 - DNA, 50 bp, Artificial Sequence
  Oligonucleotide
cctggccgctgccttcattgagtttaaagggacaggattgcccttccgtc

SEQ ID NO: 429 - DNA, 50 bp, Artificial Sequence
  Oligonucleotide
ccagcattgatctagaagcagaggaatcccagcgccttttaaaagttgtt

SEQ ID NO: 430 - DNA, 50 bp, Artificial Sequence
  Oligonucleotide
cgtggctaggcctttcctgccgagtgctctgatgcaatagtggaaatcgc

SEQ ID NO: 431 - DNA, 50 bp, Artificial Sequence
  Oligonucleotide
aaccatcccagagctggcgagaggatggagctgggtggaaactgctttgc

SEQ ID NO: 432 - DNA, 50 bp, Artificial Sequence
  Oligonucleotide
gagcagttcgctcctccctgataagagttgtcccaaagggtcgcttaagg

SEQ ID NO: 433 - DNA, 50 bp, Artificial Sequence
  Oligonucleotide
ggcagaaatgagcagttcgctcctccctgataagagttgtccaaagggtc

SEQ ID NO: 434 - DNA, 50 bp, Artificial Sequence
  Oligonucleotide
taacttccaggagttcctcattctggtgataaagatgggcgtggcagccc

SEQ ID NO: 435 - DNA, 50 bp, Artificial Sequence
  Oligonucleotide
gggcctcccttgaccccagtacgaagtctatgccctgaatccccagagta

SEQ ID NO: 436 - DNA, 50 bp, Artificial Sequence
  Oligonucleotide
cagagccccagcccctcatgtcttgccgcccttcctccatgtgtttgtaa

SEQ ID NO: 437 - DNA, 50 bp, Artificial Sequence
  Oligonucleotide
gccaaccgcctcggggcaaactcgctcttggacctggttgtctttggtcg

SEQ ID NO: 438 - DNA, 50 bp, Artificial Sequence
  Oligonucleotide
ctgtgccaccatcccgccagccattcgctcctactgatgagacaagatgt

SEQ ID NO: 439 - DNA, 50 bp, Artificial Sequence
  Oligonucleotide
gctgtctccagtgcctgttaagctgaggatacaaccaggaaatgcaacgg

SEQ ID NO: 440 - DNA, 50 bp, Artificial Sequence
  Oligonucleotide
gccacgttacatcaacacggagcatggaggcagtcaggctcgattccttt

SEQ ID NO: 441 - DNA, 50 bp, Artificial Sequence
  Oligonucleotide
gcgctgcctttcttcagcaacagaccctcaaaccaagaggaagctagatg

SEQ ID NO: 442 - DNA, 50 bp, Artificial Sequence
  Oligonucleotide
cccaggccaataagctgggtgtctaaaaggacagcttctcttccactcaa

SEQ ID NO: 443 - DNA, 50 bp, Artificial Sequence
  Oligonucleotide
gttcgtttcatcaggctctgttcctcaatggccttttgctacgtgcctcc

SEQ ID NO: 444 - DNA, 50 bp, Artificial Sequence
  Oligonucleotide
atggccatgacccagaagtatgaggagcatgtgcgggagcagcaggctca

SEQ ID NO: 445 - DNA, 50 bp, Artificial Sequence
  Oligonucleotide
gctgtagctccttggggcaaaggtactaatccctttcagcacccccactc

SEQ ID NO: 446 - DNA, 50 bp, Artificial Sequence
  Oligonucleotide
gcctgaggtgacagacagggcaggtggtaacaaaaccgttgaacctccca

SEQ ID NO: 447 - DNA, 50 bp, Artificial Sequence
  Oligonucleotide
ctgctccgacagcagccccaggaaatacgggaatggttcagggaccaagt

SEQ ID NO: 448 - DNA, 50 bp, Artificial Sequence
  Oligonucleotide
tggcctttcctacagggagctcagtaacctggacggctctaaggctggaa

SEQ ID NO: 449 - DNA, 50 bp, Artificial Sequence
  Oligonucleotide
aaggccttggactcttccctgagggttgcctgaaattccttcatgctttc

SEQ ID NO: 450 - DNA, 50 bp, Artificial Sequence
  Oligonucleotide
atcaccacactccccccagccttcacctggccatgaaggaccttttgacc

SEQ ID NO: 451 - DNA, 50 bp, Artificial Sequence
  Oligonucleotide
ggtcattggccacttcatctacagcagcctgttcccagttccctgaggcc

SEQ ID NO: 452 - DNA, 50 bp, Artificial Sequence
  Oligonucleotide
ccgtccactcctcctactgtattttattggacaggtcagactcgccgggg

SEQ ID NO: 453 - DNA, 50 bp, Artificial Sequence
  Oligonucleotide
atggagctcctggaagcagcagagtcctttgacccaggaagtgcttcagg

SEQ ID NO: 454 - DNA, 50 bp, Artificial Sequence
  Oligonucleotide
ccaccaaggccctagactcatcttggccctcctcagctccctgcctgttt

SEQ ID NO: 455 - DNA, 50 bp, Artificial Sequence
  Oligonucleotide
gcgtgggctcgattcctcagggccacgttaccacagacctgtttgtttct

SEQ ID NO: 456 - DNA, 50 bp, Artificial Sequence
  Oligonucleotide
ccccatttcatggttttcagtggcaacttactgacccttgtttttgcctg

SEQ ID NO: 457 - DNA, 50 bp, Artificial Sequence
  Oligonucleotide
ggtccccatgtgcctgttgttcagccctctctcttgttccctttctgagc

SEQ ID NO: 458 - DNA, 50 bp, Artificial Sequence
  Oligonucleotide
cacctccctgatgcctgctttcagttgagggttgggggcaatgatgagca

SEQ ID NO: 459 - DNA, 50 bp, Artificial Sequence
  Oligonucleotide
ctgctattagagcccatcctggagccccacctctgaaccacctcctacca

SEQ ID NO: 460 - DNA, 50 bp, Artificial Sequence
  Oligonucleotide
tggagcccgcgttgctgttcccacagggcctcggtttttcctaacttgct

SEQ ID NO: 461 - DNA, 50 bp, Artificial Sequence
  Oligonucleotide
gatacctgtaatcccagctacgtgggaggctgaggtgggagaattgcttc

SEQ ID NO: 462 - DNA, 50 bp, Artificial Sequence
  Oligonucleotide
ggtgggtttgcctagggacgtgtaactacaggcttttactaagccaagga

SEQ ID NO: 463 - DNA, 50 bp, Artificial Sequence
  Oligonucleotide
ggaggccagtgttgtgggcttcctgctgggactgagaaggctcacgaagg

SEQ ID NO: 464 - DNA, 50 bp, Artificial Sequence
  Oligonucleotide
acccaagtcttctcccgtccattccagtcaaatctgggctcactcacccc

SEQ ID NO: 465 - DNA, 50 bp, Artificial Sequence
  Oligonucleotide
tatgtgcagcgacccttggtgtttcccttcctcggtggctctggggtatg

SEQ ID NO: 466 - DNA, 50 bp, Artificial Sequence
  Oligonucleotide
gcctttggtcagtaatgcgttcaggagtccacaccaggcacagatggggc

SEQ ID NO: 467 - DNA, 50 bp, Artificial Sequence
  Oligonucleotide
ggcagctgtagatcttgatcttccaggtaccccatgtacctttattgagc

SEQ ID NO: 468 - DNA, 50 bp, Artificial Sequence
  Oligonucleotide
tcttggcctcccaactcttcccactcccagaatccagaagtaagctctgc

SEQ ID NO: 469 - DNA, 50 bp, Artificial Sequence
  Oligonucleotide
cgacagcaggacatacatgttggtgtgaagactgggacgacactgggtag

SEQ ID NO: 470 - DNA, 50 bp, Artificial Sequence
  Oligonucleotide
tcaacctcaccagggctgtctcttggtccacacctcgctccctgttagtg

SEQ ID NO: 471 - DNA, 50 bp, Artificial Sequence
  Oligonucleotide
ggagcagtaccttcccggagtccacgcatgtgagttgggtcaagtgcatt

SEQ ID NO: 472 - DNA, 50 bp, Artificial Sequence
  Oligonucleotide
ctctgatttttgcctctggatagtagatctcgagcgtttatctcgggctt

SEQ ID NO: 473 - DNA, 50 bp, Artificial Sequence
  Oligonucleotide
ttgccagagttttgcctgctgctttcctcgtggcctcttcttgggtagtg

SEQ ID NO: 474 - DNA, 50 bp, Artificial Sequence
  Oligonucleotide
atggaggagaccgaggaggtggctatggaggagatcgaggtggctatgga

SEQ ID NO: 475 - DNA, 50 bp, Artificial Sequence
  Oligonucleotide
gtaacggagtttagagccagggctgatgctttggtgtggccagcactctg

SEQ ID NO: 476 - DNA, 50 bp, Artificial Sequence
  Oligonucleotide
aaagaagagcaacacgattctgggatcccaggaggggaacaccatgaaga

SEQ ID NO: 477 - DNA, 50 bp, Artificial Sequence
  Oligonucleotide
gtctgcagccagcctgctcgaaactttagtcggcctgatggcttagagga

SEQ ID NO: 478 - DNA, 50 bp, Artificial Sequence
  Oligonucleotide
gagcggatgctttcatgcaccctttactgcactttctgaccaggagctac

SEQ ID NO: 479 - DNA, 50 bp, Artificial Sequence
  Oligonucleotide
cactctctgccagtggagccagaaatgacagcccaacacagataccagtg

SEQ ID NO: 480 - DNA, 50 bp, Artificial Sequence
  Oligonucleotide
cgctcgtcaccaagtctttagaatagctttagcgtcgtgaaccccgctgc

SEQ ID NO: 481 - DNA, 50 bp, Artificial Sequence
  Oligonucleotide
ctgtattaaaaaggcaaatcgaaggccgggcgcggtgactcacgcctgtc

SEQ ID NO: 482 - DNA, 50 bp, Artificial Sequence
  Oligonucleotide
cagccccctgcagctaagaattgtattgactgtcctcacagcggcttttc

SEQ ID NO: 483 - DNA, 50 bp, Artificial Sequence
  Oligonucleotide
agggctgcagggcctcccaccttccaacagacaggctctgctgtatctgt

SEQ ID NO: 484 - DNA, 50 bp, Artificial Sequence
  Oligonucleotide
gggctatgtaggcaggttaatcctccacttctcatgtggttgaaccagtg

SEQ ID NO: 485 - DNA, 50 bp, Artificial Sequence
  Oligonucleotide
cttcagtgagaaactgcccttacaaacagtcccttctctgctgtcaatcc

SEQ ID NO: 486 - DNA, 50 bp, Artificial Sequence
  Oligonucleotide
catcgcaggtaccatcaaaacggaaggcgagcatgaccctgtgacggagt

SEQ ID NO: 487 - DNA, 50 bp, Artificial Sequence
  Oligonucleotide
tcgctctggtcgcagcttaggaacagcagacgtgcactttgagcggaagg

SEQ ID NO: 488 - DNA, 50 bp, Artificial Sequence
  Oligonucleotide
gctgcaacccctcattatccaccacgcacagatggtacagctggggctga

SEQ ID NO: 489 - DNA, 50 bp, Artificial Sequence
  Oligonucleotide
gacaaaacgggcgcgatgatgccctggctttcagggtggtcagaactgga

SEQ ID NO: 490 - DNA, 50 bp, Artificial Sequence
  Oligonucleotide
gcagcgccaagcggcatccaccaagcatcaagttggagaaaagggaaccc

SEQ ID NO: 491 - DNA, 50 bp, Artificial Sequence
  Oligonucleotide
cttctctccccatcgctccacaacctgaaaccgagaaggagttgctgacc

SEQ ID NO: 492 - DNA, 50 bp, Artificial Sequence
  Oligonucleotide
ctgagaacctttcccgttactgcgttttcaccacctgtcttccccatgct

SEQ ID NO: 493 - DNA, 50 bp, Artificial Sequence
  Oligonucleotide
gctggagggcgctgtcattgtctatcagctgtactccctaatgtcctctg

SEQ ID NO: 494 - DNA, 50 bp, Artificial Sequence
  Oligonucleotide
atattccatcctgcccaacccttcctctcccatcctcaaaaaagggccat

SEQ ID NO: 495 - DNA, 50 bp, Artificial Sequence
  Oligonucleotide
tgactcaagggctgtagatgttccctttccaccccccacacttggtgcgt

SEQ ID NO: 496 - DNA, 50 bp, Artificial Sequence
  Oligonucleotide
gtagtgtatcacagtagtagcctccaggtttccttaagggacaacatcct

SEQ ID NO: 497 - DNA, 50 bp, Artificial Sequence
  Oligonucleotide
ggcatgagttagggagactgaagagtattgtagactgtacatgtgccttc

SEQ ID NO: 498 - DNA, 50 bp, Artificial Sequence
  Oligonucleotide
agtacctggggaggttagatgtgtgtttcaggcttggagtgtatgagtgg

SEQ ID NO: 499 - DNA, 50 bp, Artificial Sequence
  Oligonucleotide
cagtgcttggcacctggaagtaggtggcagatgttaacgcccttcctccc

SEQ ID NO: 500 - DNA, 50 bp, Artificial Sequence
  Oligonucleotide
ggtgaaccagaacagcacctcctcccacttaggaagctcgtgatttccag

SEQ ID NO: 501 - DNA, 50 bp, Artificial Sequence
  Oligonucleotide
ccttaatttgcaggactgccttggtggctttgtttgctgggacaaggccc

SEQ ID NO: 502 - DNA, 50 bp, Artificial Sequence
  Oligonucleotide
tattagtgcgctgtgaggtctccacccgctttgacatgggtagccttcgg

SEQ ID NO: 503 - DNA, 50 bp, Artificial Sequence
  Oligonucleotide
cagtaacgaggcttttgatgtgttgagctggaggtgagtggaccgggggc

SEQ ID NO: 504 - DNA, 50 bp, Artificial Sequence
  Oligonucleotide
tgatctcccagtcttgtccttacccattccaagtgctctgccagcccctg

SEQ ID NO: 505 - DNA, 50 bp, Artificial Sequence
  Oligonucleotide
caccaatgaatgagtccttagccctgtgtcagtttaccctcgatgccctt

SEQ ID NO: 506 - DNA, 50 bp, Artificial Sequence
  Oligonucleotide
tgtcctggcttcccctcccaaggaggatgaggatggtgcctctgaggaaa

SEQ ID NO: 507 - DNA, 50 bp, Artificial Sequence
  Oligonucleotide
ccctggggatagctggggcatttgtctagctgggctaccttctaacactt

SEQ ID NO: 508 - DNA, 50 bp, Artificial Sequence
  Oligonucleotide
tttgcccctctcaccagccgtggaagccagcagtatcgagctctcacagt

SEQ ID NO: 509 - DNA, 50 bp, Artificial Sequence
  Oligonucleotide
ctctggagtggtgtatactgccacatcagtgtttgagtcagtccccagag

SEQ ID NO: 510 - DNA, 50 bp, Artificial Sequence
  Oligonucleotide
gcatgcgtgtgcaggactggctgtgtgcttggactcggctccaggtggaa

SEQ ID NO: 511 - DNA, 50 bp, Artificial Sequence
  Oligonucleotide
agagggaaggcaggggtggaccgccatgagcatgaaaagacccgaagcaa

SEQ ID NO: 512 - DNA, 50 bp, Artificial Sequence
  Oligonucleotide
cctccagcattcagtccagggggagccacggaaaccatgttcttgcttaa

SEQ ID NO: 513 - DNA, 50 bp, Artificial Sequence
  Oligonucleotide
tcggtcctgctggaggccacgggtgccacacactcggtcccgacatgatg

SEQ ID NO: 514 - DNA, 50 bp, Artificial Sequence
  Oligonucleotide
gcctgaatgatgagcctatgtccctgcctaacactggtgtctcactcatc

SEQ ID NO: 515 - DNA, 50 bp, Artificial Sequence
  Oligonucleotide
aggctcccttctgagcctctcctgctgctgacctgatcacctctggcttt

SEQ ID NO: 516 - DNA, 50 bp, Artificial Sequence
  Oligonucleotide
ttctcctagggttatgtccagttggggtttttaaggcagcacagactgcc

SEQ ID NO: 517 - DNA, 50 bp, Artificial Sequence
  Oligonucleotide
cccagtgacgtggaagtcatcagaaccccacggtacttggagtacctctc

SEQ ID NO: 518 - DNA, 50 bp, Artificial Sequence
  Oligonucleotide
ccttggttccctaaccctaattgatgagaggctcgctgcttgatggtgtg

SEQ ID NO: 519 - DNA, 50 bp, Artificial Sequence
  Oligonucleotide
ctgatatttttccttgggggcgtaaccttcgctgaaattgctgccctgcg

SEQ ID NO: 520 - DNA, 50 bp, Artificial Sequence
  Oligonucleotide
cacttgaccccaggagacgtaggttgtggtgagctgagatcgcgccattg

SEQ ID NO: 521 - DNA, 50 bp, Artificial Sequence
  Oligonucleotide
gttctgtgtgctgtgacgactgtcaaagagtatctggccatggcggacac

SEQ ID NO: 522 - DNA, 50 bp, Artificial Sequence
  Oligonucleotide
tccagtctgtcaccctcctttcctgctcccatacacccaaggcttgtttc

SEQ ID NO: 523 - DNA, 50 bp, Artificial Sequence
  Oligonucleotide
gctaacatccattccctttcataccaccattttcaccctgtttcttcccc

SEQ ID NO: 524 - DNA, 50 bp, Artificial Sequence
  Oligonucleotide
gcgctaacattcactcttgtttgtccctggactggccatgaagtgaggag

SEQ ID NO: 525 - DNA, 50 bp, Artificial Sequence
  Oligonucleotide
cataccggctggccacgggaagcgatgataactgcgcggcattctttgag

SEQ ID NO: 526 - DNA, 50 bp, Artificial Sequence
  Oligonucleotide
ggcaccgtgtccaagtttttagaacccttgttagccagaccgaggtgtcc

SEQ ID NO: 527 - DNA, 50 bp, Artificial Sequence
  Oligonucleotide
ctcagcccgactggatcgccatctgctacaacaactgcctggagatactc

SEQ ID NO: 528 - DNA, 50 bp, Artificial Sequence
  Oligonucleotide
ggaagatgccccgacttctttggccagtgatggggaatcagtgagtgctc

SEQ ID NO: 529 - DNA, 50 bp, Artificial Sequence
  Oligonucleotide
aaaaaatgatttctggccgggcgtggtggctcaagcctgtaatcccagca

SEQ ID NO: 530 - DNA, 50 bp, Artificial Sequence
  Oligonucleotide
tggaagccctcaccaagcacttccaggactgaccagaggccgcgcgtcca

SEQ ID NO: 531 - DNA, 50 bp, Artificial Sequence
  Oligonucleotide
gggtggggtacttctccataaggcatctcagtcaaatccccatcactgtc

SEQ ID NO: 532 - DNA, 50 bp, Artificial Sequence
  Oligonucleotide
ctgcctaacgtttgcttctgtgatggttatattgcctagcaagcacaccc

SEQ ID NO: 533 - DNA, 50 bp, Artificial Sequence
  Oligonucleotide
ttgcccattcggctgtggatagagaagcaggaagagcactggacttggag

SEQ ID NO: 534 - DNA, 50 bp, Artificial Sequence
  Oligonucleotide
ctgaggcaaacaggcatgggaaaatggaagggttgaggatggaccggaga

SEQ ID NO: 535 - DNA, 50 bp, Artificial Sequence
  Oligonucleotide
ccagaatgtggtggttctgggcaacaaatgagattgtggcgacgtggaga

SEQ ID NO: 536 - DNA, 50 bp, Artificial Sequence
  Oligonucleotide
tgtgtcacagccagagggacaaagtgtgggtgatcctggagacgccagtt

SEQ ID NO: 537 - DNA, 50 bp, Artificial Sequence
  Oligonucleotide
gggatcaactgtacgcctttggtatctgaccataaagtcttttgctccgc

SEQ ID NO: 538 - DNA, 50 bp, Artificial Sequence
  Oligonucleotide
agccaaggacagagacctggaacagatgctttcattatggcctccagagg

SEQ ID NO: 539 - DNA, 50 bp, Artificial Sequence
  Oligonucleotide
ggtgctggctctctgtcacaatgcctcaaaagacatggaacccaggccta

SEQ ID NO: 540 - DNA, 50 bp, Artificial Sequence
  Oligonucleotide
ctcactatgaggaacaagagaactaggggagctgctctggtggccgtgtg

SEQ ID NO: 541 - DNA, 50 bp, Artificial Sequence
  Oligonucleotide
agtctgtagcctccccgatccaagttcctagacctcatggctgtcccctc

SEQ ID NO: 542 - DNA, 50 bp, Artificial Sequence
  Oligonucleotide
aggccttgttgcttaagacaccttcagtctttgcaggagggcatggaagc

SEQ ID NO: 543 - DNA, 50 bp, Artificial Sequence
  Oligonucleotide
ttccagcctgccagtcatgaatctcagacagcctgccacctattgccctg

SEQ ID NO: 544 - DNA, 50 bp, Artificial Sequence
  Oligonucleotide
gagctgaaagctgcggcgccactggtgccagagtcagatgtcacagatgt

SEQ ID NO: 545 - DNA, 50 bp, Artificial Sequence
  Oligonucleotide
aggcgtggtggcctgcttctgtaatcctagctagttgggaggctggcaca